(19) United States
(12) Patent Application Publication
(10) Pub. No.: US 2022/0230208 A1
(43) Pub. Date: Jul. 21, 2022

(54) SYSTEMS, DEVICES, AND METHODS FOR COMMUNICATING A WELLNESS SCORE AND/OR AN IMPROVEMENT SCORE TO A SOCIAL MEDIA PLATFORM AND OBJECTIFYING AN ONLINE REPUTATION

(71) Applicant: OutcomeMD, Inc., Los Angeles, CA (US)

(72) Inventors: Justin SALIMAN, Los Angeles, CA (US); Hooman FAKKI, Los Angeles, CA (US); Kraig van der Klomp, Santa Clarita, CA (US)

(21) Appl. No.: 17/581,312

(22) Filed: Jan. 21, 2022

Related U.S. Application Data
(60) Provisional application No. 63/140,234, filed on Jan. 21, 2021.

(51) Int. Cl. G06Q 30/02; G16H 50/30; G16H 10/20; G06Q 50/00

(52) U.S. Cl. CPC G06Q 30/0282; G06Q 50/01; G16H 10/20; G16H 50/30

(57) ABSTRACT

Patients may conveniently and efficiently share wellness and/or improvement scores with a social media platform and/or online review platform in response to receipt of an automatic prompt following completion of a medical questionnaire used to determine the respective wellness and/or improvement score. This may allow the patient and/or individual within an online social network of the patient to monitor his or her progress with, for example, a treatment, a recovery from a treatment, and/or a wellness program. Additionally, or alternatively, a review of a treatment provider of the patient that includes a wellness and/or improvement score provides useful, measured, data to others who may be reading the online review and/or searching for a treatment provider who may provide treatments known to improve a wellness of a patient.

### RELATED APPLICATION

The present application is a NON-PROVISIONAL, of and claims priority to, U.S. Patent Application No. 63/140,234 filed on 21 Jan. 2021 and entitled “Systems, Devices, And Methods For Communicating A Wellness Score To A Social Media Platform And Objectifying An Online Reputation,” which is incorporated in its entirety herein.

### FIELD

The present invention relates to medical information technology. More specifically, the present invention relates to systems, devices, and methods for communicating a wellness score to a social media platform and managing an online reputation.

### BACKGROUND

Current medical and healthcare systems lack objective treatment outcome measures. As a consequence, healthcare providers and patients are often uncertain of which treatments are effective, and healthcare providers cannot be judged by their true effectiveness at treating patients. This inability to universally measure medical treatment effectiveness, combined with the widespread availability of subjective online reviews of providers, has resulted in physicians focusing on patient catering rather than patient care because they cannot risk the patient submitting a negative online review that will damage his or her reputation. A consequence of this is that clinicians over utilize healthcare resources and over prescribe tests and medications to make patients happy because their business models are dependent on receiving good subjective reviews from patients rather than delivering good end results. This phenomenon has been associated with increased overall US healthcare costs and increased mortality.

### SUMMARY

Systems, devices, and methods for communicating a wellness and/or improvement score to a social media platform and/or online review platform for publication thereon are herein disclosed. The social media platform may be any social media platform that accepts posts and/or publishes content from one or more users of the platform. Exemplary social media platforms include FACEBOOK™, TWITTER™, LINKEDIN™, and REDDIT™. The social media platform may provide private and/or public social media accounts that are associated with one or more users or groups of users, such as a patient (or group of patients) diagnosed with a diagnosis and/or undergoing wellness and/or medical treatment and/or a provider of a wellness and/or medical treatment. The online review platform may be any review platform offered online, or via the Internet, that provides a tools to users that enable them to prepare and post, or publish, online reviews of, for example, providers of wellness and/or medical treatments. Examples include, but are not limited to, YELP™, HEALTHGRADES™, etc. At times, performance of one or more processes described herein may be responsive to receipt of an indication that the patient has scheduled the treatment and/or an encounter with a treatment provider who may provide the treatment to the patient.

In some embodiments, measures of a patient's wellness and/or improvement may be achieved via scoring answers a patient provides to a medical questionnaire that assesses, for example, how a patient is recovering from a treatment and/or coping with a diagnosis. In some cases, the medical questionnaires may be instruments for wellness assessment that are validated by a scientific community (e.g., American Medical Association and/or Food and Drug Administration) so that the questions and answers thereto are scientifically valid measures of a treatment, or wellness program, outcome.

In some embodiments, the methods disclosed herein may be performed via providing a patient with a medical questionnaire via a first graphic user interface (GUI) displayed on a display device that may be included in, for example, a smart phone, computer, or tablet computer. The medical questionnaire may be associated with a sender (e.g., a medical treatment provider, a doctor, a treatment facility, a coach and/or wellness treatment provider), a treatment and/or a medical condition (e.g., diagnosis) of, and/or issue of concern (e.g., an undiagnosed medical condition) to, patient. The medical questionnaire may be associated with a scoring procedure for scoring responses to the medical questionnaire to obtain, for example, the wellness score. In some instances, medical questionnaire may be provided to the patient via a patient account that may be associated with the sender and/or the treatment provider. The patient account may be associated with information that includes the social media platform and/or online review platform preference of the sender.

A set of responses to the medical questionnaire may be received from the patient via the first GUI and the scoring procedure may be applied to them to determine a wellness score for the patient. This wellness score may be compared with a previously determined wellness score for the patient and an improvement score may be determined based on a result of the comparison. In some embodiments, an indication of the determined wellness score and/or the improvement score may be provided to the patient via, for example, the display device.

Often times, the medical questionnaire used to receive a plurality of sets of responses over time (and therefore determine a corresponding plurality of wellness scores) may be the same so that the set of responses used to determine wellness scores used to determine an improvement score are responsive to the same, or a similar, medical questionnaire. In some cases, the determination of an improvement score may be based upon a comparison of a presently determined wellness score with one or more previously determined wellness scores that, on some occasions, may span a duration of time (e.g., a week, a month, a year, etc.) measured from, for example, a time when the presently determined wellness score is determined. In some instances, the previously determined wellness score may be determined by applying the scoring procedure to a set of previously received responses to the medical questionnaire. Additionally, or alternatively, the previously determined wellness score may be determined by applying the scoring procedure to a set of previously received responses to the medical questionnaire, wherein the set of previously received responses to the medical questionnaire are be received prior to the patient receiving the treatment.

In some embodiments, it may be determined whether the improvement score is above a threshold for improvement scores and, if so, the patient may be provided with a second GUI by which the patient may provide an instruction to post the improvement score to an account on a social media platform and/or online review platform responsively to a determination that the improvement score is above the threshold. In some cases, the threshold may be specific to and/or defined by, for example, a treatment facility, and/or a provider of the treatment. The instruction to post the improvement score on the social media platform and/or online review platform may be received via the second GUI. The account on the social media platform may be public and/or private and may be a social media account of the patient, the sender of the medical questionnaire, a treatment provider, and/or a treatment facility. On some occasions, the second GUI may include a plurality of social media posting options and an indication of a social media preference of the patient may be received via the patient's interaction with the second GUI. Additionally, or alternatively, in some instances, a social media preference and/or online review platform preference of the patient may be received via a query of the patient and/or a device the patient is using to provide the set of answers (e.g., smart phone or tablet computer) and, in these instances, the plurality of social media and/or online review posting options provided by the second GUI may be responsive to the received social media and/or online review platform preference of the patient.

In some embodiments, a social media preference and/or online review platform preference of a sender of the medical questionnaire and/or a treatment provider who administered the medical treatment to the patient may be received and the social media platform and/or online review platform posting options provided by the second GUI may be responsive to the received social media preference and/or online review platform of the treatment provider. At times, the social media preference and/or online review platform preference of a sender may be embedded in the medical questionnaire and/or provided by a software application providing the medical questionnaire to the patient.

On some occasions, the second GUI may provide the patient with a plurality of types of social media platforms and/or online review platforms to choose from and the instruction to post the improvement score on the social media platform and/or online review platform may include a selection of the social media platform and/or online review platform from the plurality of social media platforms and/or online review platforms the patient wishes to post the improvement score to. In these instances, the formatting of the improvement score for posting to the account on the social media platform and/or online review platform may be specific to the selected social media platform and/or online review platform.

The improvement score may then be formatted for posting to the social media account on the social media platform and/or online review platform. Often times, the formatting of the improvement score may generate a formatted social media post that includes the improvement score. In some cases, the formatting of the improvement score may include encoding the social media post with a hyperlink linking the formatted social media post to, for example, a uniform resource locator (URL) associated with the sender of the medical questionnaire. Additionally, or alternatively, the formatting of the improvement score may include encoding the social media post with metadata that, for example, associates the formatted social media post with the sender of the medical questionnaire, the treatment the patient has received, a treatment facility, a third party, a diagnosis of the patient, and/or a level of improvement of the patient. Additionally, or alternatively, the formatting of the improvement score may include encoding the social media post with data and/or instructions (e.g., login information, security certificates, permissions, etc.) for accessing and/or posting to a private social media account of an entity and/or individual who is not the patient (e.g., a treatment provider, a caregiver, a treatment facility, etc.). Additionally, or alternatively, the formatting of the improvement score may include encoding the social media post with formatting instructions (e.g., color, font, icon content and/or placement of icons or message, etc.).

In some cases, information and/or instructions used to perform the formatting of the improvement score may be included in and/or associated with the medical questionnaire and/or a software program providing the medical questionnaire, wellness scores, and/or improvement scores to the patient. For example, the medical questionnaire may be embedded with metadata and/or instructions that facilitates communication with, for example, the social media platform, the online review platform, and/or an account thereon. Additionally, or alternatively, the second GUI and/or instructions used to render the second GUI on the display device may be embedded with information (e.g., metadata and/or instructions) that facilitates communication with, for example, the social media platform, online review platform, and/or an account thereon.

Once formatted, the formatted social media post that includes the improvement score may then be communicated and/or posted to the social media platform and/or online review platform. At times, the patient may be provided with an indication (e.g., acknowledgement message) that the improvement score has been successfully posted on the social media platform and/or online review platform.

In some embodiments, a characteristic of the patient may be received and it may be further determined whether the patient characteristic impacts the determination of at least one of the wellness score and the improvement score and, if so, a value of the wellness score and/or the improvement score may be adjusted based upon the patient characteristic. In these embodiments, the determination of whether the improvement score is above the threshold for improvement scores may include determining whether the adjusted improvement score is above a threshold for adjusted improvement scores. Exemplary patient characteristics include, but are not limited to, demographic information (e.g., age, gender, etc.) and whether the patient has a comorbidity and/or confounding injury and/or diagnosis.

In some embodiments, the patient may be provided with a third GUI by which the patient may provide an instruction to post the improvement score to an account on the social media platform and/or online review platform. At times, provision of the third GUI may be responsive to a determination that the improvement score is above a threshold for improvement scores. An instruction to post the improvement score to the social media platform and/or the online review platform may be received and the improvement score may be formatted for posting to the the social media platform and/or online review platform. Then, the improvement score may be communicated to the the social media platform and/or the online review platform for posting thereon.

In some embodiments, a template social media post and/or online review that includes the improvement score may be provided to the patient prior to posting the improvement score on the social media platform and/or online review platform and an instruction to post the template social media post and/or template online review to the respective social media platform and/or online review platform may include acceptance and/or modification of the respective template social media post and/or online review. Additionally, or alternatively, a template social media post/online review that includes the improvement score may be provided to the patient prior to posting the improvement score on the social media platform and/or online review platform. A modification to the template social media post/online review may be received and a modified social media post/online review may be generate responsively to the received modification. An instruction to post the modified social media post/online review to a respective social media platform and/or online review platform may then be received and the improvement score may be communicated to the social media platform and/or online review platform using the respective modified social media post/online review.

### BRIEF DESCRIPTION OF DRAWINGS

The present invention is illustrated by way of example, and not limitation, in the figures of the accompanying drawings in which:

FIGS. 1A and 1B are block diagrams of exemplary systems for generating and/or presenting a medical portal interface, in accordance with some embodiments of the present invention;

FIG. 2 provides a flowchart illustrating a process for determining a threshold improvement score, in accordance with some embodiments of the present invention;

FIG. 3 provides a flowchart illustrating a process for providing a user with an interface by which he or she may communicate a wellness and/or improvement score via an email, message, and/or a social media post, in accordance with some embodiments of the present invention;

FIG. 4 is a flowchart of an exemplary process for determining an adjusted improvement score for the patient, in accordance with some embodiments of the present invention;

FIG. 5 provides a flowchart of an optional process that may be executed following a determination that the improvement score is below a threshold, in accordance with some embodiments of the present invention;

FIGS. 6A and 6B provide a flowchart of an optional process that may be executed following a determination that the improvement score is above the threshold, in accordance with some embodiments of the present invention;

FIG. 7 provides a flowchart illustrating an exemplary process for preparing an enriched social media post and/or message, in accordance with some embodiments of the present invention;

FIG. 8A is a screen shot of an exemplary GUI that displays a question of a medical questionnaire, in accordance with some embodiments of the present invention;

FIGS. 8B-8G are exemplary graphic user interfaces (GUIs) that display a wellness score and/or the improvement score, in accordance with some embodiments of the present invention;

FIG. 9A is a screen shot of an exemplary user interface showing a template email that provides a wellness score, in accordance with some embodiments of the present invention;

FIG. 9B is a screen shot of an exemplary user interface showing a template email that provides an improvement score, in accordance with some embodiments of the present invention;

FIG. 9C is a screen shot of an exemplary GUI that provides an initial wellness score along with a button that may facilitate copying of the pop-up window content so that it may be pasted into an email or message.

FIG. 9D is a screen shot of an exemplary GUI that provides a pop-up window that includes template email content

FIGS. 10A-10F provide screen shots of exemplary GUIs that include different exemplary social media posts, in accordance with some embodiments of the present invention;

FIGS. 11A-11D provide screen shots of exemplary GUIs that include different exemplary online review posts, in accordance with some embodiments of the present invention; and

FIG. 12 is a block diagram showing a system, in accordance with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### Written Description

A patient who is planning to undergo and/or is actively undergoing, for example, a medical treatment, a wellness treatment, and/or an athletic and/or wellness (e.g., weight loss, smoking cessation, meditation for stress relief, etc.) training program may be provided with one or medical questionnaires that are each associated with a scoring procedure. As used herein, a “patient” may be, for example, a person diagnosed with a medical condition, a person undergoing a medical and/or wellness treatment and/or a caregiver of the patient. The medical questionnaire(s) may be provided by, for example, a software program running on a processor that may be resident in, for example, a user electronic device (e.g., smart phone or laptop computer) and/or a cloud computing platform. When responses to the one or medical questionnaires are received by the processor, they may be scored using the scoring procedure to determined a wellness score. In many cases, a baseline wellness score may be determined prior to commencement of the treatment and/or training program. This baseline (or a previously determined) wellness score may then be compared with, or otherwise evaluated in regard to, one or more later (weeks, months, or years) determined wellness score(s) to determine an improvement score. The improvement score may be an indication of how the patient's condition and/or progress with the treatment and/or training program has changed over time. At times, an improvement score of a patient my be negative number, which may indicate that the patient's wellness scores are decreasing in value over time.

In some embodiments, the patient may be automatically prompted by the processor to share wellness scores and/or improvement scores with a social media platform and/or online review platform via, for example, a GUI that provides one or more options for the creation and/or posting of a message that includes the improvement score and/or wellness score. At times, this prompting may occur when, for example, the patient has a wellness score and/or improvement score that is above a threshold value. The threshold value may be set by, for example, the patient, a treatment provider, and/or a treatment facility. If the patient decides he or she would like to share a wellness score and/or improvement score with a social media platform and/or online review platform, the patient may communicate same to the respective social media platform and/or online review platform. At times, a prompt for sharing a wellness and/or improvement score may be a template social media post and/or online review platform post that includes, for example, the wellness and/or improvement score, the medical condition and/or treatment being monitoried and/or a provider of the treatment.

In some embodiments, the processes described herein may be performed in a similar manner (e.g., same medical questionnaire(s)) for a large (e.g., 100-500,000) number of patients who receive the same, or similar treatments and/or have the same and/or similar diagnosis and/or comorbidities so that, for example, measuring improvement and/or treatment outcomes may be standardized across populations of patients. This standardization may offer an ability to study and/or correlate positive outcomes with, for example, treatment types and/or treatment providers for large numbers of people.

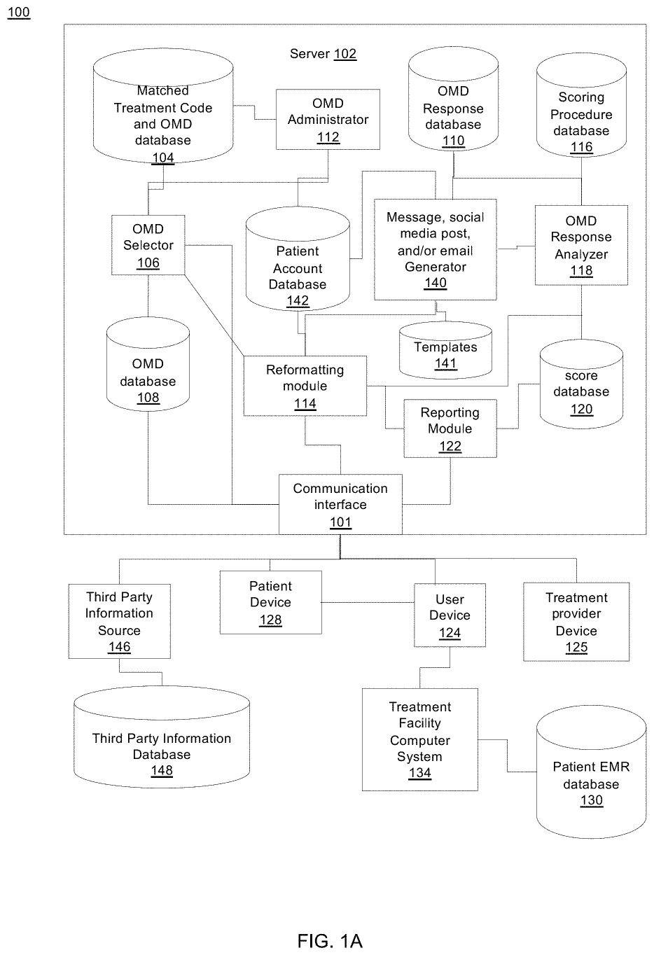

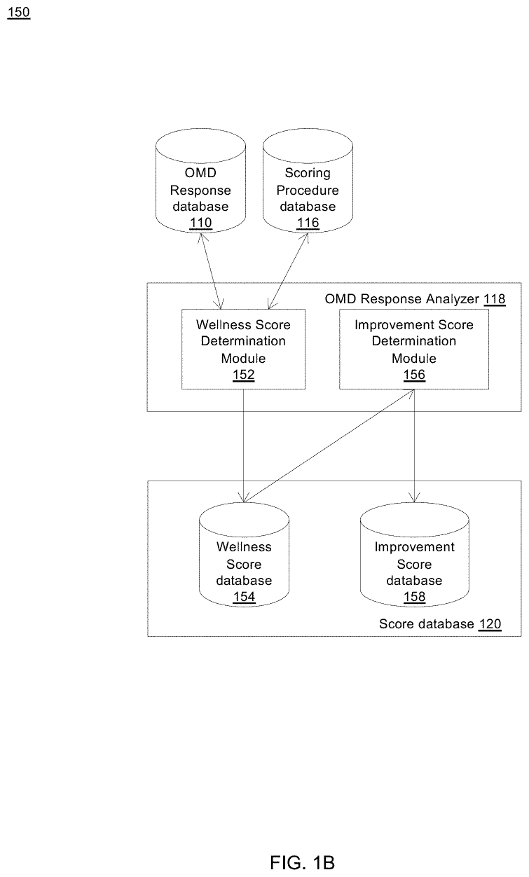

Turning now to the figures, FIG. 1A provides a block diagram of an exemplary system 100 that may be used to execute one or more of the processes described herein so that a user and/or patient may communicate a wellness and/or improvement score to a social media platform, a messaging platform, and/or to another individual or group of individuals (e.g., a support group) via email. At a high level, system 100 includes a server 102, a patient device 128, a user device 124, a treatment provider device 125, and a treatment facility computer system 134, all directly or indirectly communicatively coupled to one via a network like the Internet and/or a private network. Patient device 128 may be any device (e.g., a smartphone, a laptop computer, a tablet computer, a desktop computer, etc.) that enables communication between a patient and other components of system 100. Similarly, user device 124 and/or treatment provider device 125 may be any device (e.g., a smartphone, a tablet computer, a laptop computer, a desktop computer, etc.) that enables communication between a treatment provider (also referred to herein as a “user”) and other components of system 100. In some cases, treatment provider device 124 may also be a device that is enabled to perform a specific healthcare treatment and/or diagnostic task. For example, treatment provider device 125 may be a network-connected treadmill, a network connected blood pressure monitor, or a network-connected ultrasound machine. For simplicity, only one treatment provider device 125 is depicted, while it is understood that in practice there may be a plurality of treatment provider devices 125. Similarly, while only one patient device 128 is depicted, it is understood that in practice there may be a plurality of patient devices 128, one or more for each patient.

Treatment facility computer system 134 may be a computer system that is located in, and/or communicatively coupled to, a treatment facility (i.e., a computer/server that is located in a doctor's office or treatment facility). As is understood in the art, an EMR (as stored in EMR database 130) may include notes prepared by a treatment provider regarding the health of a patient, results of medical tests performed on a patient, treatments administered on a patient, etc. Further due to HIPAA regulations, medical records from treatment facility computer system 134 may be communicated to user device 124, treatment provider device 125, patient device 128, and/or server 102 using one or more security protocols that may be compliant with HIPAA requirements. It is understood that other data (i.e., not patient-specific data) may be transmitted between user device 124, patient device 128, treatment provider device 125, sever 102, and/or facility computer system 134 via a conventional communication network (e.g., the Internet, a wired network, a wireless network, a private network, a public network, routers, switches, etc.), which has not been depicted in FIG. 1A. Further, it is understood that user device 124, patient device 128, treatment provider device 125, server 102, and/or facility computer system 134 may be communicatively coupled to via a communication network (e.g., the Internet), a private network, and/or a blockchain.

In one embodiment, any one of the components of system 100 may replace any patient identifying information (e.g., patient name, social security number, birthdate, address, etc.) in medical records and/or a patient account with, for example, a binary string to form anonymized medical records containing no patient identifying information (e.g., patient name, social security number, birthdate, address, etc.). More generally, any patient identifying information in medical data (e.g., EMR, questionnaire responses provided by a patient, wellness scores computed for a patient, etc.) may be replaced with a binary string to form anonymized and/or de-identified medical data. Such anonymized medical data may be stored at, for example, server 102, treatment facility computer system 134, patient device 128, and/or user device 124, in various databases operated by server 102 (e.g., OMD response database 110, score database 120, etc.), cloud-based storage (e.g., Amazon Web services, Google Cloud platform or Microsoft Azure) (not depicted), etc. In the event the anonymized medical data is intercepted by a malicious individual (e.g., a hacker), patient privacy may still be preserved since the malicious individual will not be able to associate the anonymized medical data with any specific patient. Additionally, or alternatively, information pertaining to one or more thresholds for an improvement score, adjustments to a threshold for an improvement score, and/or characterisitics to associate with adjustments to a threshold for an improvement score may be stored in score database 120.

A mapping between respective binary strings and respective patient identifying information may be securely stored (e.g., stored in an encrypted manner) at one or more components of system 100. Such mapping may enable an electronic device (e.g., server 102, user device 124, treatment provider device 125, and/or patient device 128) to access medical data associated with a specific patient. The steps for an electronic device to access the medical data of a patient may proceed as follows. First, the electronic device may be authenticated by HIPAA compliance server (e.g., the electronic device is required to provide the proper credentials, such as a login identifier and password). Following successful authentication, the electronic device may request medical data concerning an exemplary patient, John Doe. For example, server 102 may map the patient name of “John Doe” to “patient 001010” via the mapping and/or indexing, and the medical data of patient 001010 may be retrieved from a database which stores the anonymized medical data (e.g., OMD response database 110, score database 120, etc.).

In one embodiment, the process flow for system 100 may proceed as follows. Upon server 102 receiving a request from, for example, user device 124 and/or treatment provider device 125, patient device 128, and/or server 102 may provide an outcome measurement device (OMD) to the patient, treatment provider, and/or user device 124, 125, and/or 128. An OMD may be a modality, instrument, or tool by which medical information about a patient may be collected that may also be referred to herein as a “medical questionnaire”. Exemplary OMDs include, but are not limited to, a medical questionnaire, a physical test of the patient (e.g., blood test, physical examination, or blood pressure), and a patient reported outcome (PRO) instrument. At times, an OMD may referred to as a medical questionnaire herein. In some cases, the request to administer the OMD may be triggered via the entry of a treatment code (e.g., a Current Procedural Terminology (CPT) code) or a treatment/diagnostic test name into the patient's EMR (as stored in patient EMR database 130), a treatment facility's billing software, and/or a treatment facility's scheduling software. In some instances, a request to administer and OMD may be triggered by a patient requesting receipt of an OMD via, for example, his or her wellness account and/or a request to administer an OMD may be triggered by a patient who requests to send an OMD to a friend or colleague via, for example, a link to an OMD and/or an invitation to respond to an OMD.

In some instances, when a treatment/diagnostic test name or other related information (other than a treatment/diagnostic code) is received, server 102 may interpret (using, for example, natural language analysis) the treatment/diagnostic test name so that it matches one or more treatment codes. In such cases, OMD selector 106 may determine one or more OMDs that match the treatment code via matched treatment code and OMD database 104. More generally, matched treatment code and OMD database 104 may also include matches between treatment names and OMDs, as well as diagnostic codes and OMDs when selecting OMDs for delivery to a patient device 128 and/or user device 124.

Next, OMD selector 106 may retrieve the one or more determined OMDs from OMD database 108. The retrieved OMDs may be provided to OMD administrator 112, which may administer the OMDs to the patient via, for example, patient device 128 and/or user device 124. In the instance that the retrieved OMDs are patient reported outcome (PRO) instruments, the PRO instruments may be provided to patient device 128. Completed OMDs (also called OMD responses) may be transmitted from patient device 128 (and may be received as a set of responses) and stored in OMD response database 110. More specifically, OMD responses may be stored in OMD response database 110 in an anonymized fashion. For example, OMD responses may be indexed in OMD response database 110 by a binary string, or other anonymous identifier, rather than by a patient name. Similarly, to the discussion above, if an OMD response for a specific patient is desired, the patient name may be mapped to a binary string by, for example, server 102, and the OMD response associated with that binary string may be retrieved from OMD response database 110.

OMD response analyzer 118 may analyze the OMD responses stored in OMD response database 110 to generate one or more scores (e.g., a wellness score, an improvement score, etc.). Such scores are described in more detail below with regard to FIG. 1B. Such analysis may rely upon scoring procedures stored in scoring procedure database 116. Such scoring may also rely upon other considerations and/or esoteric factors 132 stored at patient EMR database 130. In most circumstances, what may be referred to herein as “other considerations” are factors that may directly, or closely, relate to and/or have an impact on, a medical condition, diagnosis, and/or treatment. For example, it is known that smoking has an impact on a person's cardiovascular health. Thus, whether a person smokes may be an “other consideration” for a patient's treatment related to cardiovascular health. This relationship between cardiovascular health and smoking may be indexed or otherwise stored in other consideration database 132. An esoteric factor is one that is less directly related to a medical condition, diagnosis, and/or treatment but may still have an impact thereon. For example, a vegetarian diet may have an impact on a person's cardiovascular health, yet this impact may be less well understood when compared with the impact of smoking on the same patient's cardiovascular health. As such, a person's status as a vegetarian may be considered an “esoteric factor.”

The scores that are generated by OMD response analyzer 118 may be stored at score database 120. More specifically, scores may be stored in score database 120 in an anonymized fashion so as to, for example, comply with HIPAA regulations or other data security requirements/preferences. For instance, wellness scores associated with a patient may be indexed by a binary string in score database 120 rather than by a patient name.

Finally, reporting module 122 may report the scores to one or more of user device 124, patient device 128, treatment provider device, 125, and/or treatment facility computer system 134. In addition to the request for a treatment, there are other events that may prompt an OMD to be administered to a patient. In one example, the scheduling of an initial appointment (e.g., a consultation) for a patient to discuss a medical condition with a healthcare professional may prompt an OMD to be administered to the patient. Administering an OMD to the patient prior to this initial appointment may be useful for establishing a baseline state of health for the patient, but the selection of the OMD may have some complexity, as no treatment code, treatment name, or diagnostic code may yet be available when the initial appointment is scheduled because, for example, a diagnosis has not been made. In many instances, all that the patient will provide is a brief description of the symptoms he/she may be experiencing (e.g., shortness of breath, fever, etc.) and/or a chief complaint. In one embodiment, such symptoms may be provided to OMD selector 106, which attempts to match the symptoms with one or more treatment codes, treatment names, or diagnostic codes and select a corresponding OMD. Such matching by OMD selector 106 may be performed using a learning machine and/or artificial intelligence. For instance, matches between, for example, symptoms and treatment codes; symptom and treatment names; and/or symptoms and diagnostic codes may be provided by a healthcare professional when treating patients, and such matches may be used to train a model that can then be used to determine treatment codes, treatment names or diagnostic codes based on, for example, a patient's symptoms and/or treatment provider notes. Upon determining a treatment code, treatment name, or a diagnostic code, OMD selector 106 may select one or more OMDs based on matches provided in matched treatment code and OMD database 104 (as described above). It is anticipated that the determination of a treatment code, treatment name or diagnostic code by OMD selector 106 may be, in some instances, an imperfect process, so a healthcare provider, or other expert, may be asked to make any necessary adjustments to the treatment code, treatment name and/or diagnostic code determination, before OMD selector 106 selects the one or more OMDs.

In the examples provided above, it was assumed that an OMD is administered to a patient via patient device 128. In other instances, a medical professional may be required to administer the OMD to the patient. For example, server 102 may notify user device 124 and/or treatment provider device 125 that one or more OMDs should be administered as part of, for example, a medical examination of a patient. In one example, if a patient has recently undergone cardiothoracic surgery, OMD administrator 112 may provide one or more OMDs to user device 124 (e.g., the Intrathoracic Gas Volume Test, Total Lung Capacity Test, Vital Capacity Test, 6 Minute Walk Test, Aortic Insufficiency Test, Mitral Regurgitation Test and/or Aortic Valve Area Test) that could, or should, be administered to the patient during an exam and/or provide one or more mechanisms to user device 124 (e.g., fillable forms) for the treatment provider to enter the OMD responses.

Server 102 may further include a patient account database 142, a message, social media post, and/or email generator 140, a template database 141, a reformatting module 114, a communications interface 101, a third-party information source 146, and a third-party information database 148. Communication interface 101 may facilitate communication between server 102 and an external device such as third-party information source 146, patient device 128, and/or user device 124. In some embodiments, communication interface 101 may resemble communication interface 1118. Exemplary information that may be received from patient account database 142 includes, but is not limited to, patient identifiers, demographic information for one or more patients, patient-entered information (e.g., adverse life events, medication/treatment compliance, answers to OMDs, supplemental treatments, etc.), treatment history, historical OMD responses, wellness scores, improvements scores, and so on.

Third-party information source 146 may be any source of information that is not the patient and/or operated by and/or directly associated with a user of user device 124 (e.g., treatment facility staff or medical professionals) and/or an entity operating server 102. Examples include pharmacies, medical treatment facilities other than medical treatment facilities coupled to treatment facility computer system 134 and/or patient EMR database 130 (e.g., laboratories, radiologists, chiropractors, physical fitness facilities, etc.), medical device retailers, and other service providers for patients such as ride-share services that may be able to provide information regarding pick-up and drop-off times for patients at a facility that may administer medical treatment. Third-party information database 148 may store information relevant to one or more patients who may, or may not, have a patient account that may be stored in patient account database 142. Exemplary information stored in third-party information database 142 includes, but is not limited to, pharmaceutical refill information (e.g., dates refills were dispensed, type and/or quantity of pharmaceuticals dispensed, etc.), purchases made at/via the third-party information source 146 (e.g., supplements, braces, durable medical goods, etc.), clinic information (e.g., when appointments were scheduled, whether patient arrived for appointment, notes from a treatment provider regarding an encounter with a patient, etc.) and so on.

Patient accounts may be associated with each individual patient under, for example, the care of a particular treatment facility. Information about a patient may be associated with and/or stored along with patient account information. Information about a patient may come from a plurality of sources including, but not limited to, the patient, a treatment provider, a user of a server providing access to a patient account, and a third-party (e.g., an insurance company, a treatment provider not associated with the treatment facility, and/or a pharmacy).

Patient accounts may be generated at/by server 102 responsively to instructions from the patient (as provided via, for example, patient device 128) and/or responsively to a user like a treatment facility administrator or medical treatment provider providing instructions via, for example, user device 124.

FIG. 1B depicts one embodiment of a system 150 that supports the operation of OMD response analyzer 118 and score database 120 (and some associated components). OMD response analyzer 118 may comprise wellness score determination module 152. In one embodiment, wellness score determination module 152 retrieves responses to an OMD from OMD response database 110, and further may retrieve a scoring procedure associated with the OMD responses from scoring procedure database 116. The scoring procedures may be indexed by, for example, an identifier of an OMD, for which responses have been received, making for easy retrieval of a corresponding scoring procedure. Various scoring procedures may be employed to score a completed OMD, and in one embodiment, the generated score may be known as a “wellness score”. In some cases, a “wellness score” may serve to indicate how severe a patient's symptoms are. In these cases, a low wellness score may indicate that a patient's symptoms are relatively more severe than a higher wellness score such that a subsequent higher wellness score indicates an improvement (i.e., decrease in severity) in the symptoms.

In the case where an OMD is a questionnaire (or PRO instrument), a certain weighting may be used to score or evaluate the patient's responses. For example, certain responses that are more objective in nature (e.g., heart rate, blood glucose level, etc.), may receive greater weights (and hence have a greater influence on the wellness score) than certain responses that are more subjective in nature (e.g., degree of pain, mood, etc.). The reverse scenario, of course, could be true in which subjective responses receive a greater weight than objective responses (e.g., fatigue or mental illness). Scores generated by wellness score determination module 152 may be stored in wellness score database 154. The wellness scores may be indexed in various fashions, for ease of retrieval. In one embodiment, wellness scores may be indexed according to one or more of a patient identifier (e.g., binary string to protect patient privacy), medical condition, treatment provider, treatment facility, time at which OMD was completed, etc.

Improvement score determination module 156 may retrieve two wellness scores for a patient (e.g., a first score calculated for an OMD completed at a first time point and a second score calculated for an OMD completed at a second time point) from wellness score database 154. Improvement score determination module 156 may calculate the difference between the first and second score, and such difference may be known as an improvement score. The improvement score may be stored in improvement score database 158. In one case, a relative improvement score may be calculated as the improvement score (i.e., the difference described above) normalized by a maximum improvement score, which may be calculated based on, for example, other considerations 132 stored in a patient's EMR. The maximum improvement score may take into consideration other factors such as the state of a patient prior to a medical treatment (e.g., if patient was in fairly good health, the maximum improvement score might be lower than if the patient was in poor health), and/or the age of a patient (e.g., younger patients might have a higher maximum improvement score than older patients), etc. An improvement score (or a relative improvement score) may be stored in improvement score database 158. The improvement scores may be indexed in various fashions, for ease of retrieval. In one embodiment, improvement scores may be indexed according to one or more of a patient identifier (e.g., binary string to protect patient privacy), medical condition, treatment provider, treatment facility, and time duration over which improvement score was measured, etc.

The components and/or databases of systems 100, 150, and/or 103 of FIGS. 1A and/or 1B may be a series of one or more components (e.g., computers, servers, databases, etc.) that may, in some instances, be geographically disparate.

As disclosed herein, a wellness score and/or an improvement score for one or more aspects of the patient's medical condition and/or physiological systems may then be determined by scoring responses to one or more assessments, which in some cases may be patient reported outcome (PRO) assessments that have been validated to assess a patient's medical condition via medical literature and/or accepted best practices within the medical field. In some embodiments, determination of a wellness score may include querying a scoring database like scoring procedure database 116, for a scoring metric and/or scoring procedure associated with the medical questionnaire provided in step 305. In some instances, this querying may include retrieving a scoring procedure from scoring procedure database 116 using an identifier of the medical questionnaire. For instance, a medical questionnaire may be associated with a code (e.g., 3232) and this code may be used to retrieve a scoring procedure from scoring procedure database 116. Example scoring procedures include taking an average of all the patient responses (e.g., assuming all responses are numeric), taking a weighted average of the patient responses (e.g., weighting certain responses higher than other responses), adjusting the range of patient responses (e.g., changing responses choices from 2, 3, 3 to 1, 4, 6). In some embodiments, determining a wellness score may include retrieval of a sub-scoring procedure that may be specific to the patient (i.e., associated with the patient's account and/or a comorbidity of the patient) as may be indicated by, for example, the patient's account and/or EMR. The scored responses may then be used to determine a wellness score associated with the received responses and/or a sub-set of received responses.

An improvement score (or percentage change) may be a determination of how a patient's condition has changed over time. In some embodiments, determination of an improvement score may involve comparing (e.g., averaging, subtracting, determining a percentage change, determining a time weighted average, etc.) one or more previously determined wellness scores with a currently determined wellness score in order to determine how a patient's wellness score has changed over time (e.g., 3 weeks, 3 months, 1 year, etc.).

Message, social media post, and/or email generator 140 may be configured to generate one or more of a social media post, message, and/or email as described herein according to one or more processes described herein via communication with one or more components of system 100 such as score database 120, OMD response database, scoring procedure database 116, reporting module 122, and/or system 150. In some embodiments, message, social media post, and/or email generator 140 may be in communication with template database 141 to, for example, extract template messages, emails, and/or social media posts stored thereon.

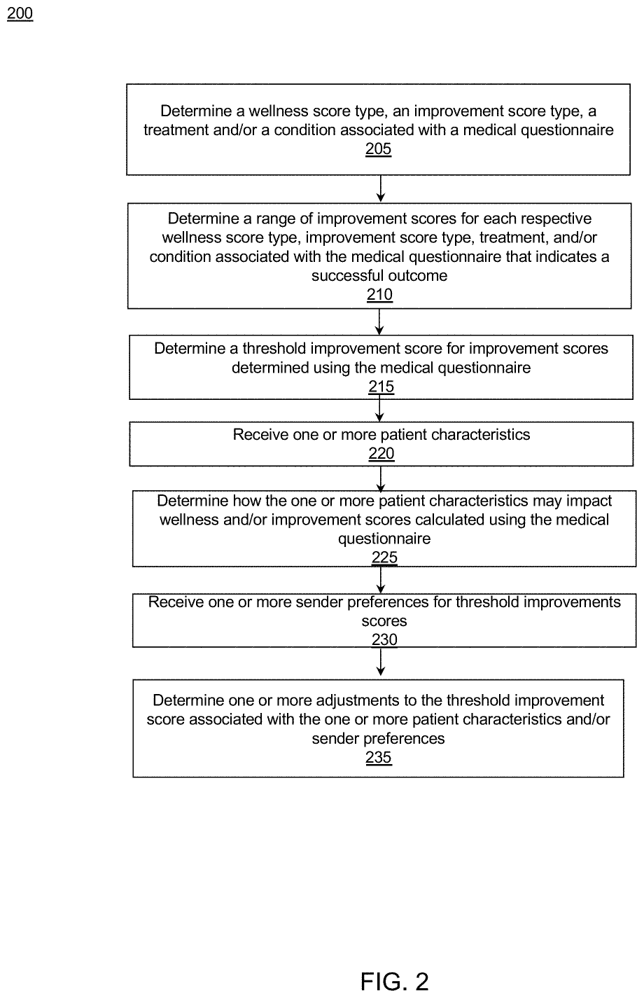

FIG. 2 provides a flowchart illustrating a process for determining a threshold improvement score. Process 200 may be executed by, for example, any of the systems and/or system components disclosed herein.

In step 205, a type of wellness score, a type improvement score, a treatment, and/or a condition associated with a medical questionnaire and/or a group of medical questionnaires used to determine a wellness and/or improvement score may be determined. In some embodiments, types of wellness scores, types of improvement scores, treatments and/or conditions received in step 205 may be categorized according to, for example, severity, impact to the general health and wellness of patient, typical persistence of a medical condition, expected amounts (e.g., full, 75%, 50%) of recovery, and/or expected rates of recovery (days, weeks, months, years).

In step 210, a range of improvement scores for each respective wellness score type, improvement score type, treatment, and/or condition associated with the medical questionnaire that indicates a successful outcome may be determined. A successful outcome may be an improvement score with an absolute value (e.g., 10% and/or 10 points) in all circumstances regardless of treatment and/or condition, an improvement score with an absolute value in particular circumstances (e.g., an improvement score of 10% for a cortisone shot treatment for a spinal injury or an improvement score of 90% for recovery from cataract surgery). Additionally, or alternatively, a successful outcome may be relative to, for example, the wellness score type, improvement score type, treatment, condition, and or timeline for recovery from a condition or treatment.

In step 215, a threshold improvement score for improvement scores determined using the medical questionnaire may be determined. How this threshold improvement score may be used is discussed below. A threshold improvement score may be an absolute value (e.g., 10%) in all circumstances regardless of treatment and/or condition, an absolute value (e.g., 10% or 30%) in particular circumstances (e.g., a low threshold for treatments known to have long recovery timelines or limited degrees of recovery as with a chronic illness e.g., an improvement score of 10% for a cortisone shot treatment for a spinal injury or an improvement score of 90% for recovery from cataract surgery). Additionally, or alternatively, a threshold improvement score may be relative to, for example, the wellness score type, improvement score type, treatment, condition, and or timeline for recovery from a condition or treatment.

Additionally, or alternatively, a threshold improvement score may be set following statistical analysis of a plurality of wellness and/or improvement scores taken over a range of time for a plurality of patients so that, for example, statistically determined trajectories for improvement for various medical conditions, diagnoses, and/or treatments may be determined and therefore used to set improvement score thresholds for various medical questionnaires.

In some embodiments, setting a threshold for an improvement score associated with a medical questionnaire may be responsive to a timeline for treatment and/or recovery from a treatment for patient. It is sometimes the case that patients improve from some treatments more rapidly immediately following the treatment and then continue to improve over time at a slower rate. For example, when recovering from a knee replacement surgery, a patient may experience rapid recovery between an initial wellness score taken the day after the surgery (e.g., not being able to put any pressure on the affected leg immediately following the treatment) and a wellness score determined four weeks following the surgery (e.g., able to walk on crutches). The same patient may be expected to experience a slower rate of recovery a few months following the treatment so an improvement score determined using wellness scores at four and five months following surgery may be lower because the change in the patient's mobility and pain when comparing month four and five in recovery may be less significant than it was in the first month following the treatment but still within an expected range of recovery for the treatment at the time following treatment. Thus, an improvement score threshold set for the first four weeks following a knee replacement surgery may be higher than an improvement score threshold set that measures improvements between the patient's condition between the fourth and fifth months following the surgery because the expected rate of recovery (i.e., improvement) between fourth and fifth months following the surgery is lower than the expected rate of recovery (i.e., improvement) between the day after the surgery to one month following the surgery.

In some cases, full recovery from a medical condition, such as a chronic illness (e.g., asthma, chronic obstructive pulmonary disease (COPD), arthritis, diabetes, dementia, Parkinson's disease), is not possible and treatment is administered overtime to manage symptoms of the underlying medical condition. In these cases, “improvement” of wellness scores taken overtime may be minimal and, in some instances, maintaining a consistent or slowly decreasing wellness score and/or improvement score and/or keeping a wellness score and/or improvement score within a particular range may be the desired, or best possible, outcome. In these instances, an improvement score and/or a wellness score threshold may be set accordingly. For example, when managing chronic illness and/or terminal illness for a patient, slowing with the decline of the patient's medical condition may be the desired, or best outcome. In these cases, an improvement score threshold that is a low number (e.g., −10%, −5%, or 0) may be set.

Additionally, or alternatively, a threshold improvement score may be set following statistical analysis of a plurality of wellness and/or improvement scores taken over a range of time for a plurality of patients so that, for example, statistically determined, or average, trajectories for improvement for various medical conditions, diagnoses, and/or treatments may be determined and used to set improvement score thresholds for various medical questionnaires and/or types of wellness and/or improvement scores.

Optionally, in step 220, one or more patient characteristics (e.g., demographic information, comorbidities, medications the patient is taking, etc.) may be received and it may be determined how the received patient characteristic(s) may impact wellness and/or improvement scores calculated using the medical questionnaire (step 225). Then, one or more adjustments to the threshold improvement scores associated with the one or more patient characteristics may be determined (step 235). Additionally, or alternatively, in some embodiments one or more sender preferences for a threshold improvement score may be received (step 230) and these thresholds may be incorporated into a determined threshold improvement score (step 235). Additionally, or alternatively, execution of step 235 may include determining one or more adjustments to a threshold improvement score associated with one or more patient chracterisitics and/or sender preferences. Following step 235, the one or more adjustments to the threshold improvement score and/or the associations between the adjustments and the patient characterisitics and/or sender preferences may be saved in a database like score database 120.

In some embodiments, execution of process 200 may end following execution of step 215. However, at times, steps 220 and 225 and/or steps 230 and 235 may be performed to determine one or more adjustments to the improvement score threshold. In some cases, these adjustments may be case (e.g., patient or sender) specific.

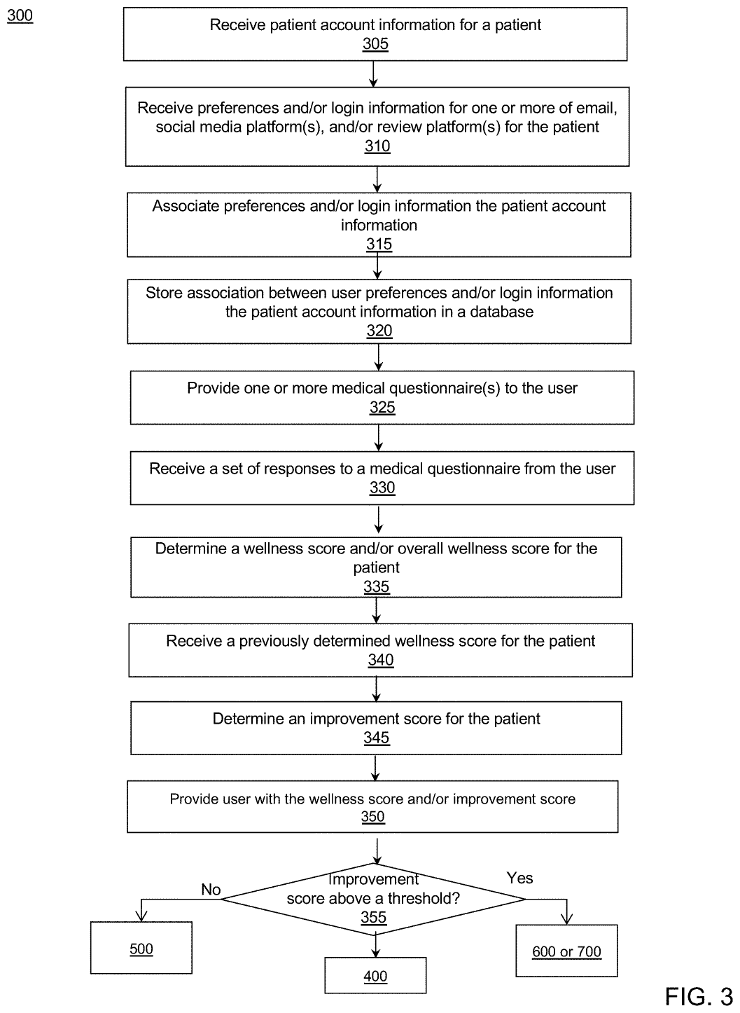

FIG. 3 provides a flowchart illustrating a process 300 for providing a user with an interface by which he or she may communicate a wellness and/or improvement score via an email, message, and/or a social media post. Process 300 may be executed by, for example, any of the systems and/or system components disclosed herein.

In step 305, patient account information may be received from a user. Exemplary users include, but are not limited to, the patient associated with the patient account, a caregiver for the patient, a public relations professional working with/for the patient, and/or a medical treatment/care provider for the patient. The patient account information may be, for example, patient account login information (e.g., biometric data, a username, and/or a password) and/or patient account administrator information, which may also be, for example, biometric data, a username, and/or a password. In some embodiments, the patient account information that may be received may include one or more social media, email, and/or messaging preferences. Social media preferences may include, for example, preferred social media platforms, user names, privacy settings, groups or subscriber lists, preferred posting places, greetings, branding, logos, template post content, formatting preferences, etc. Exemplary email preferences include, but are not limited to, email platform to be used (e.g., GMAIL™, OUTLOOK™, etc.), addressee information (e.g., email addresses), template email content, template email formatting, branding, logos, template email content, formatting preferences, etc. Exemplary messaging preferences include, but are not limited to, a type of message (e.g., SMS text message), text messaging platform (e.g., iMESSAGE™, WHATSAPP™, etc.), text messaging phone numbers, addressee information (e.g., phone number), template message content, template message formatting, branding, logos, template message content, formatting preferences, etc.

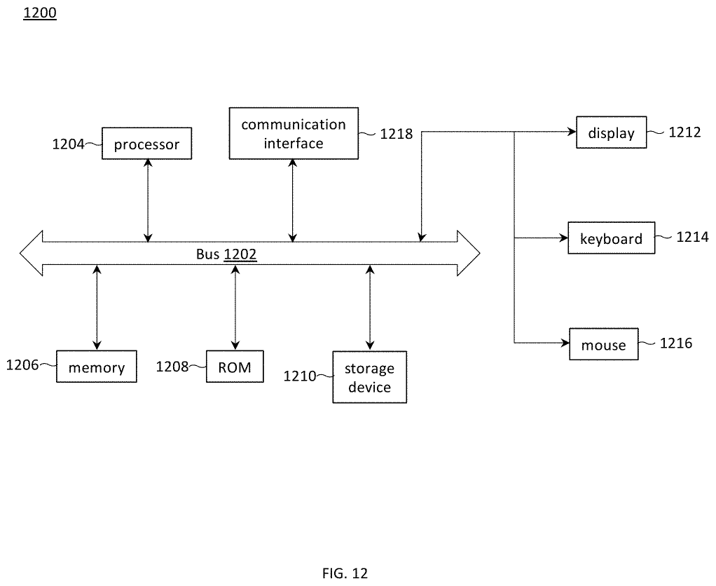

In some embodiments, the patient account may be set up by, and/or associated with (e.g., linked to and/or in communication with) a treatment provider who provides one or more treatments to the patient. The patient and/or user may interact with his or her patient account via, for example, a software application running on a personal electronic device (e.g., smart phone or tablet computer) such as patient device 128 and/or user device 124. The software application may be configured to facilitate communication with the patient and/or user via, for example, a display device and communication interface (e.g., touch-sensitive display, keyboard, etc.). Further details about the hardware used to facilitate communication between the software program the patient/user and the treatment provider are provided below with regard to FIG. 12.

Optionally, in step 310, user and/or patient preferences and/or login credentials (e.g., user name and password) for one or more social media platforms, social media accounts, email software programs, email accounts, online review platforms, and/or online review accounts may be received. Exemplary preferences include, but are not limited to, preferred text or symbols (e.g., emoji, logos, or graphic elements) to be included in social media posts, email, and/or online review, treatments the patient has received, patient diagnosis, and a length of time the patient has been undergoing a treatment. On some occasions, the user/patient preferences may be used to customize template emails, messages, social media posts, and/or online reviews that are provided to the patient/user for their approval prior to communication to the appropriate email recipient, social media account, and/or online review platform/account, respectively.

In step 315, the preferences received in step 310 may be associated with the patient account running on the patient/user electronic device and/or on a server such as server 102 and stored (step 320) in a memory like memory 1206, ROM 1208, storage device 1210, and/or patient account database 142.

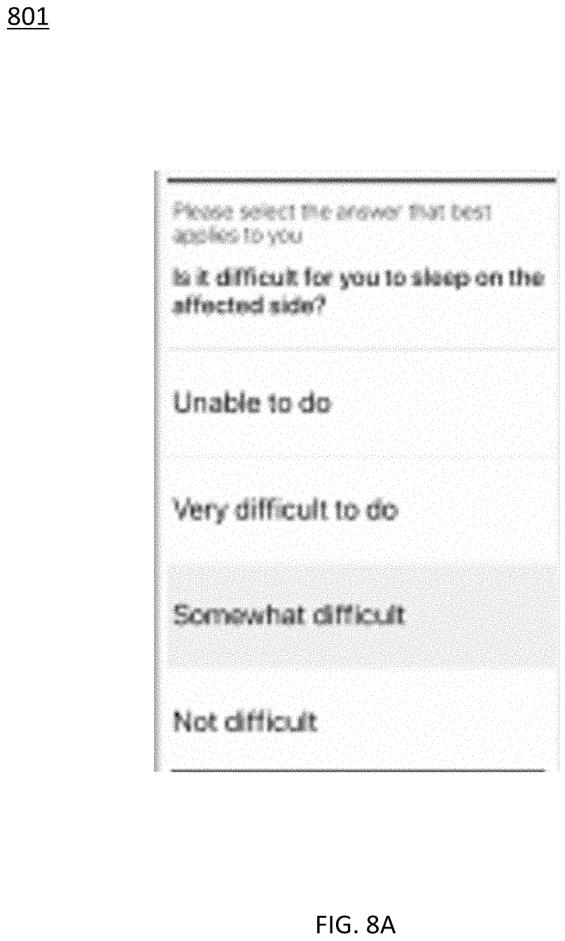

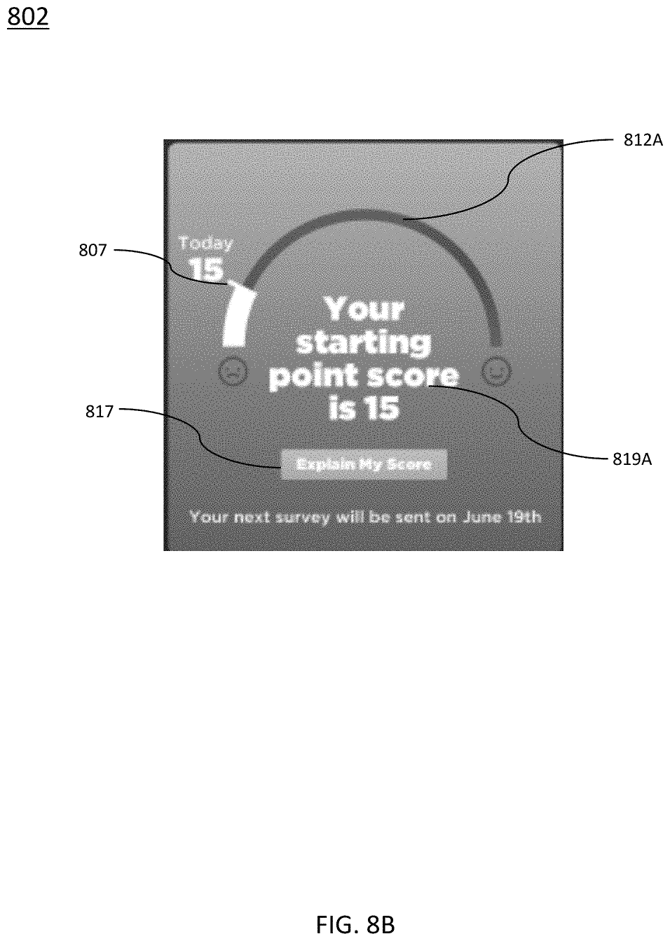

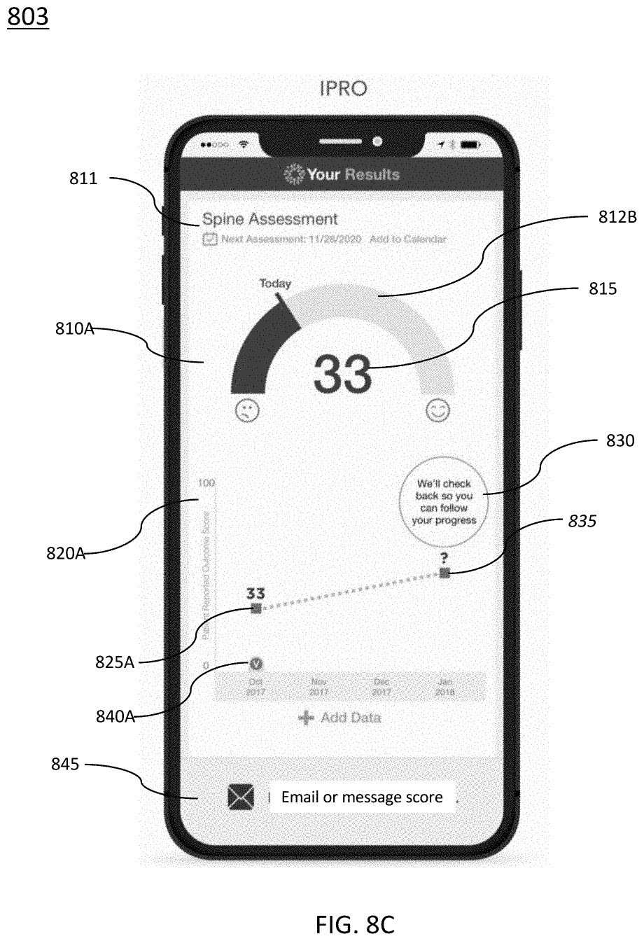

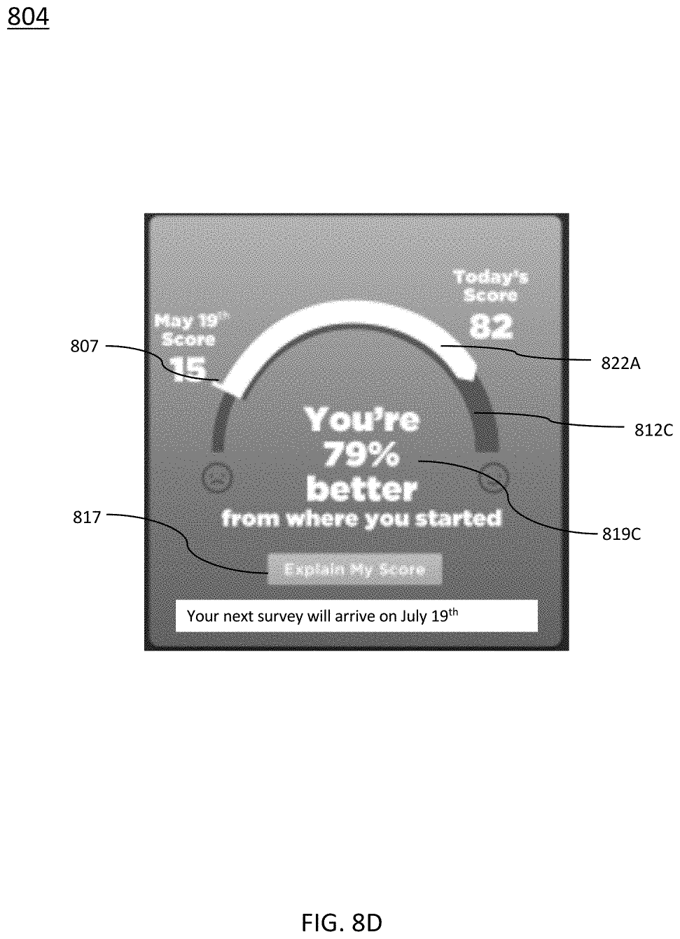

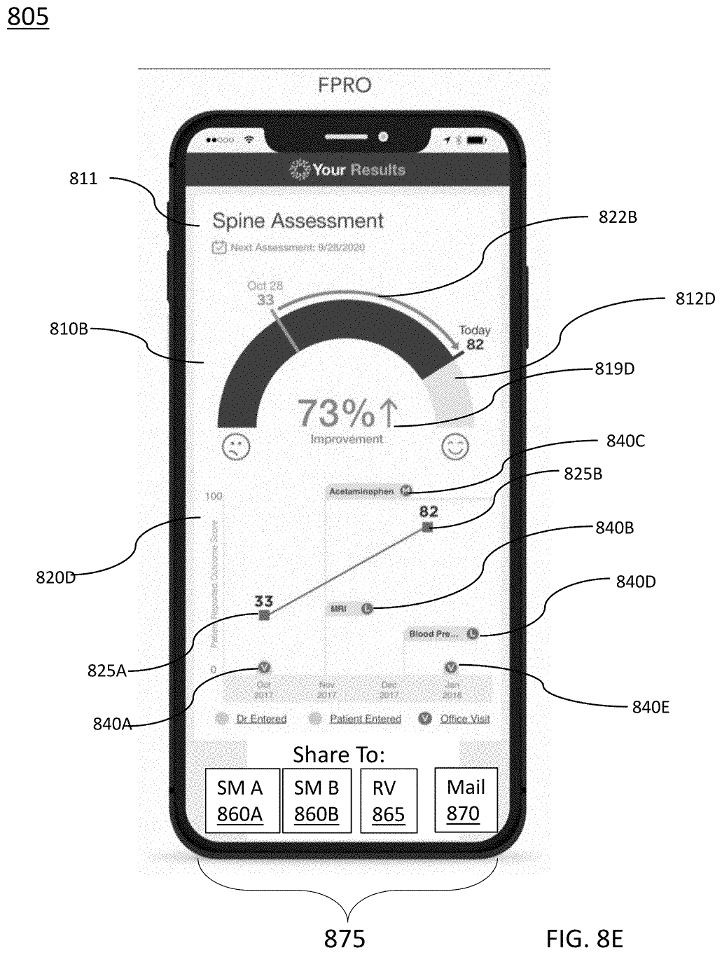

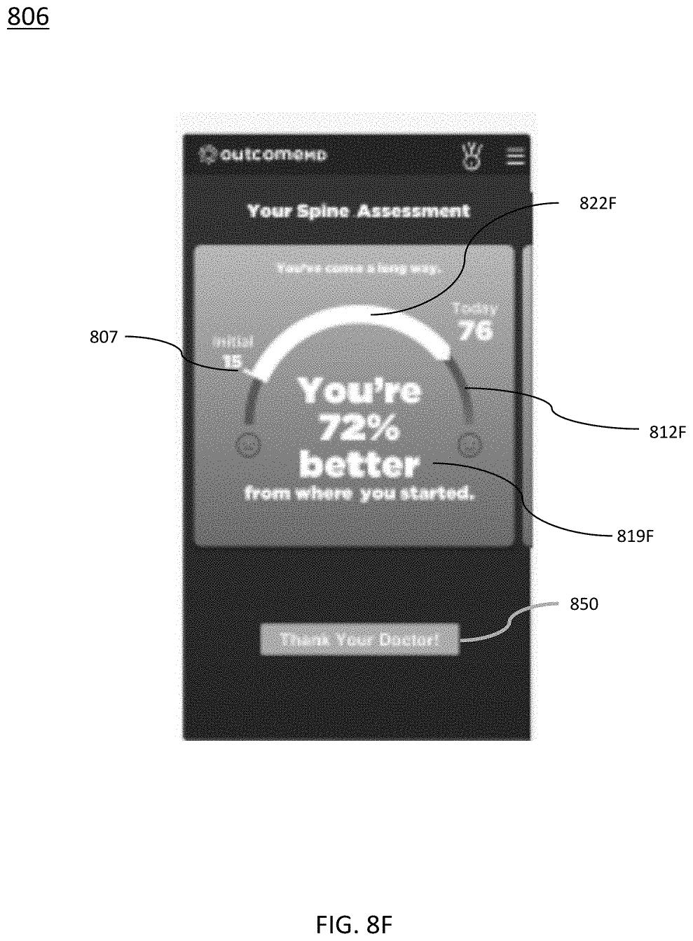

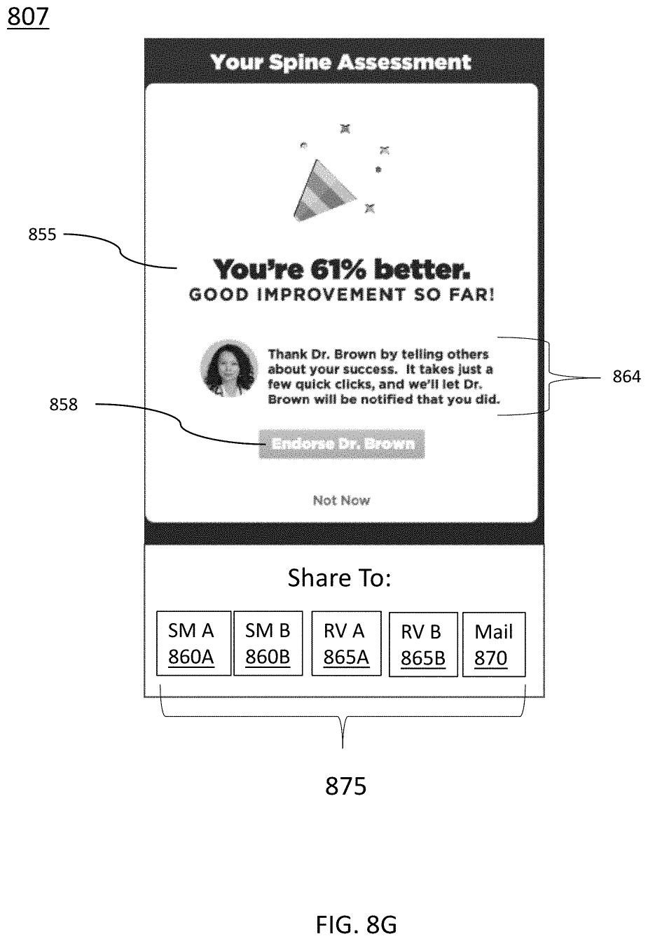

In step 325, one or more OMDs and/or medical questionnaires (OMDs and medical questionnaires may be collectively referred to herein as a “medical questionnaire”) may be provided to the patient/user. In some instances, the electronic device is owned and/or operated by the patient or user as their personal electronic device. In other instances (e.g., when the patient or user does not have a personal electronic device), the one or more medical questionnaires may be provided to an electronic device operated by, for example, a medical facility or physician. On some occasions, step 325 may be executed by the software program running on the personal electronic device the patient/user is using and the medical questionnaire may be provided to the user by way of a graphic user interface by which the patient and/or user may view the medical questionnaire(s) and provide responses thereto. An exemplary graphic user interface that provides a question included in a medical questionnaire provided to the patient is provided by graphic user interface 801 of FIG. 8A, which provides a question (in this case “is it difficult for you to sleep on the affected side”) and a series of answers the patient/user may select (in this case “unable to do,” “very difficult to do,” “somewhat difficult,” and “not difficult”). In the example of FIG. 8A, the answer of “somewhat difficult” has been selected.

In some embodiments, step 325 is executed responsively to an action by, for example, the patient, user, and/or treatment provider as a way to, for example, gather information about the patient's primary complaint or medical concern, the patient's general state of health, the patient's wellness with regard to one or more medical conditions and/or treatments, and/or the patient's improvement (or lack thereof) with regard to one or more medical conditions and/or treatments. Exemplary actions include, but are not limited to, scheduling of a treatment to be performed on/by the patient, scheduling of an encounter (e.g., telehealth and/or in-person visit) with the treatment provider, and/or requesting a change in treatment (e.g., when the patient requests a change in medication and/or administration of a treatment).

In step 330, a set of responses to the one or more medical questionnaire(s) may be received by, for example, a processor like processor 1204 and/or server like server 102 via, for example, direct entry of responses into an interface, or GUI, provided in step 325 and, in some embodiments, communication of the set of responses from, for example, a patient device like patient device 128, user device 124, and/or a treatment provider device like treatment provider device 125 to the server.

A wellness score and/or an improvement score for one or more aspects of the patient's medical condition and/or physiological systems may then be determined (step 335). In some embodiments, determination of a wellness score may include querying a scoring database like scoring procedure database 116, for a scoring metric and/or scoring procedure associated with the medical questionnaire provided in step 325. In some instances, this querying may include retrieving a scoring procedure from scoring procedure database 116 using an identifier of the medical questionnaire that may, in some cases, be associated with, for example, the medical questionnaire and/or patient account. For instance, a medical questionnaire may be associated with a code (e.g., 3232) and this code may be used to retrieve a scoring procedure from scoring procedure database 116 and a scale (e.g., 1-5, 1-40, 1-85) for the scoring and/or normalizing of the medical questionnaire. Exemplary scoring procedures include, but are not limited to, calculating an average of all the patient responses, calculating a weighted average of the patient responses (e.g., weighting certain responses higher than other responses), and/or adjusting a scale of patient responses. In some instances, where, for example, a plurality of medical questionnaires are provided to the user, a score for each medical questionnaire may be determined and these scores may be combined into a single wellness score (referred to herein as an overall wellness score) by, for example, normalizing a scale for scoring each of the medical questionnaires to a single scale (e.g., 1-10 or 1-100) and averaging or applying a weighted average to the plurality of wellness scores that are determined.

In some embodiments, a single wellness score determined in step 335 may combine the scoring two (or more) different medical questionnaires, which for the purpose of this example will be labeled MQ1 and MQ2 respectively. Determining the single wellness score may involve scoring MQ1 and MQ2 using their respective scoring procedures, normalizing a scale of the score for MQ1 and MQ2 to be on a single scale of, for example, 1-100, and then averaging the normalized scores for MQ1 and MQ2. Alternatively, the score for MQ1 and MQ2 may be normalized to be on a single scale of, for example, 1-50 and then the score for MQ1 and MQ2 may be added together to generate a wellness score on a 1-100 scale.

Additionally, or alternatively, execution of step 335 may include retrieval of a sub-scoring procedure that may be specific to the patient (e.g., associated with the patient's account, a diagnosis of the patient, or a comorbidity of the patient) as may be indicated by, for example, the patient's wellness account and/or EMR. The scored responses may then be used to determine an overall wellness score associated with the received response and/or a sub-set of received responses.

Then, in step 340, a previously determined wellness score for the patient may be received via, for example, querying memory 1206, storage device 1210, the patient account, patient account database 142, and/or an EMR database such as patient EMR database for one or more of the patient's previously determined wellness scores. In some embodiments, the received previously determined wellness score may be an initial pre-treatment wellness score. Additionally, or alternatively, when, for example, there are a plurality of previously determined wellness scores for the patient, the queried for/received previously determined wellness score may be the most recent previously determined wellness score, and/or an average of a set (2, 4, 8, 10, 20, etc.) of previously determined wellness scores.

For embodiments where more than one wellness score or sub-score was determined in step 335, a query for a wellness score may include a request for multiple types of previously-determined wellness scores that were incorporated into the overall wellness score determined in step 335 so that the components of the overall wellness may be collectively and/or individually received and/or analyzed.

In step 345, an improvement score for the patient may be determined by, for example, comparing the wellness determined in step 335 with the previously determined wellness score received in step 340 so that a change therebetween may be determined. This change may represent how a patient's condition has changed (e.g., improved and/or declined) over time. For example, in some embodiments, determination of an improvement score may involve comparing (e.g., averaging, subtracting, determining a percentage change, determining a time weighted average, etc.) the received previously determined wellness scores with wellness score determined in step 335 in order to determine how a patient's wellness score has changed over time (e.g., 3 weeks, 3 months, 1 year, etc.). The improvement score may be, for example, a percentage change in wellness scores determined at different points in time and/or a numerical value.

Continuing with the example above, in embodiments where the determination of a single wellness score in step 335 includes scoring two different medical questionnaires, execution of step 345 may include determining a change in each of the scores MQ1 and MQ2 between a presently and previously determined score for MQ1 and MQ2 respectively and/or determining a change in the presently and previously determined single wellness scores.

In step 350, the user may be provided with the wellness score and/or the improvement score via, for example, communicating same to a patient device like patient device 128 via a user interface like graphic user interfaces (GUIs) 802, 803, 804, 805, 806, and/or 807 of FIGS. 8B, 8C, 8D, 8E, 8F, and 8G, respectively. GUI 802 of FIG. 8B provides a window that provides a message 819A that includes a wellness score (in this case, 15) for the patient following his or her completion of a medical questionnaire. GUI 802 also provides a first semi-circular graph 812A that graphically depicts a range of scores from 0-100 on which a wellness score graphic element 807 is superimposed showing that a starting wellness score for the patient is 15. GUI 802 further includes an icon 817, the selection of which may trigger an explanation of the wellness score.

Graphic user interface 803 provides a wellness score of 33 to a patient who has taken a spine assessment medical questionnaire. The type of assessment taken, as well as a date for when the next assessment is scheduled, is shown in a heading 811. Graphic user interface 803 includes a first wellness window 810A that provides a message 815 that includes a wellness score of 33, that may have been determined in step 335, shown as a numerical value in first wellness window 810A and also as a value on a graphic object such as a second semi-circular graph 812B. Second semi-circular graph 812B may also provide a marker (in this case a line extending outward from the semicircular graphic) indicating where in the range of 1-100 the patient's wellness score falls when the assessment is taken today.

Graphic user interface 803 also provides a first scatter plot graph 820A that includes a scatter plot showing time measured in months on the X-axis as a function of wellness, or outcome, scores on the Y-axis (shown as patient reported outcome score on the Y-axis of graph 820A), wherein a first point 825A, which represents the wellness score of 33 determined “today,” is aligned with October 2017 and the next scheduled provision of a medical questionnaire is scheduled for January 2018, which is represented by a square with a question mark above it 835 along with a message bubble 830 stating “We'll check back so you can follow your progress.” First scatter plot graph 820A also includes a visual indicator 840A for when an office visit has occurred, which also corresponds to October 2017. In some cases, graphic user interface 803 may be a graphic user interface that is initially displayed to the user when he or she takes an initial medical questionnaire and/or has a first wellness score for a particular type of assessment determined. As such, a graphic user interface like graphic user interface 803 may be displayed to a user when a patient's improvement score cannot be determined because there is no previously determined wellness score to compare the wellness score determined today (i.e., the wellness score of 33) to.

GUI 804 provides a third message 819C that includes the patient's improvement score (in this case, 79%) along with a third semi-circular graph 812C on which an initial wellness score 807 of 15 is shown along with a first improvement score graphic element 822A that is superimposed on third semi-circular graph 812C that graphically depicts a range on the graph from the initial wellness score of 15 to today's score of 82.

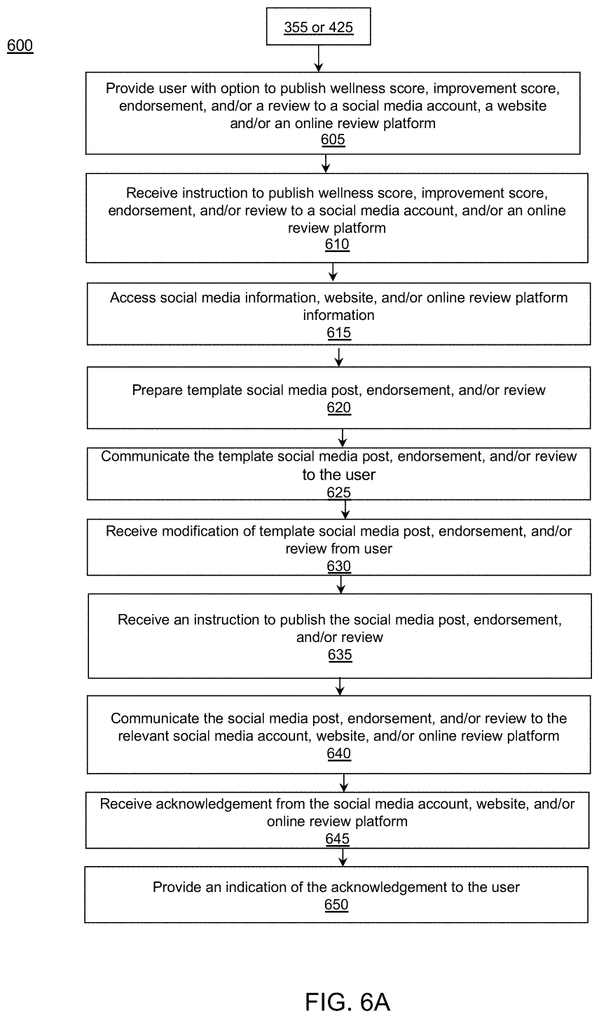

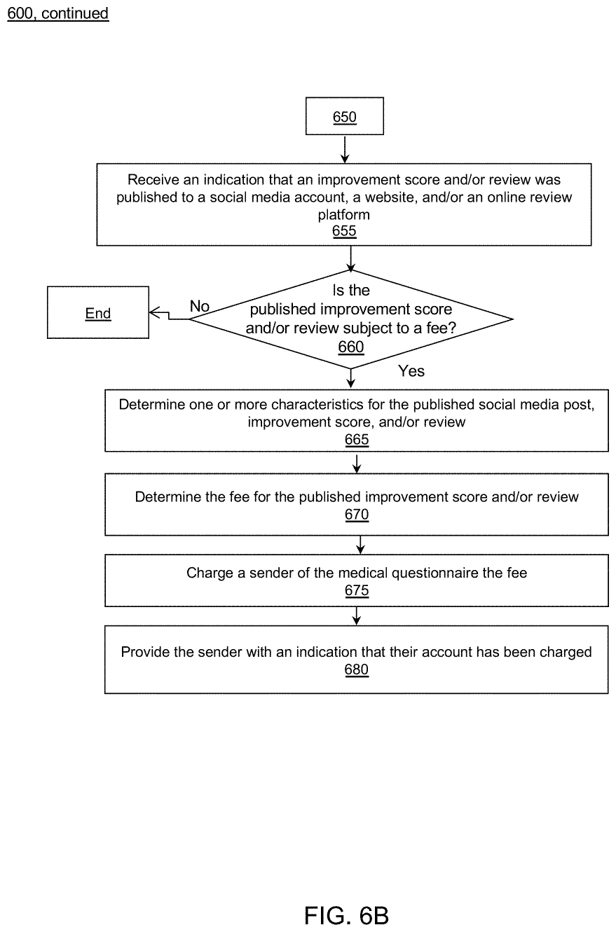

In step 355 of process 300, it may be determined whether an improvement score determined in step 345 is above a threshold. In some embodiments, the threshold improvement score may be determined via execution of process 200 or a portion thereof. Exemplary improvement score thresholds include, but are not limited to, 0% (indicating no improvement), 10%, 20%, or 25% and, in some cases, may be set by an associate of the patient (e.g., employer and/or public relations professional), a clinician, treatment center, and/or medical administrator who, in some cases, may be have requested that the user be provided with the medical questionnaire in step 325. In some instances, the threshold may be referred to herein as an endorsement threshold and, at times, when the improvement score is above the endorsement threshold, a user may be prompted to provide an endorsement of, for example, a medical treatment provider, a medical treatment facility, a treatment, and/or a course of action (e.g., diet or exercise program) as explained below with regard to, for example, process 600 shown in FIGS. 6A and 6B. Additionally, or alternatively, the threshold may be referred to herein as an endorsement pop-up threshold and, at times, when the improvement score is above the endorsement pop-up threshold, a user may be prompted to provide an endorsement of, for example, a medical treatment provider, a medical treatment facility, a treatment, and/or a course of action (e.g., diet or exercise program) via a pop-up window or GIU that is provided to the user via an interface by which he or she is, for example, entering answers to a medical questionnaire and/or viewing wellness score and/or an improvement score, as explained below with regard to, for example, process 600 shown in FIGS. 6A and 6B.

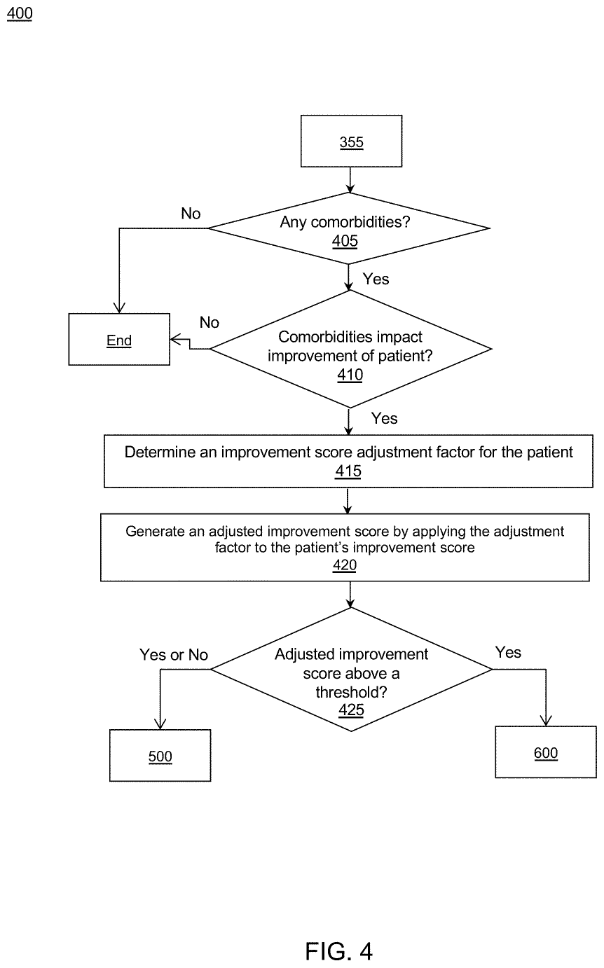

FIG. 4 is a flowchart of an exemplary process 400 for determining an adjusted improvement score for the patient. Process 400 may be executed by any of the systems or system components disclosed herein. Optionally, process 400 may be executed following execution of step 355 regardless of whether the improvement score is above a threshold. However, optional process 400 is more likely to be executed when it is determined in step 355 that the improvement score is below the threshold.

In step 405, it may be determined whether the patient has any comorbidities by, for example, asking the patient whether or not he or she has any comorbidities and/or querying patient and/or patient account information that may be stored in, for example, memory 1206, storage device 1210, patient account database 142, and/or patient EMR database 130 for information regarding patient comorbidities. Exemplary comorbidities include, but are not limited to, a diagnosed medical condition, an injury, and an adverse life event (e.g., death in the family, divorce, job loss, etc.). When the patient does not have any comorbidities, process 400 may end. When the patient does have a comorbidity, it may be determined whether that comorbidity impacts and ability for an improvement in a medical condition the patient is diagnosed with and/or recover from a treatment the patient has received (step 410) and, if so, an improvement score adjustment factor for the patient's improvement may be determined (step 415).

The adjustment factor may be, for example, a percentage and/or a statistical weight that is applied to what are more factors in the calculation of the patient's improvement score and/or the overall determined improvement score for the patient. Execution of step 410 may include determining whether the comorbidity is relevant to the patient's diagnosis and/or treatment received. For example, if the patient has the comorbidity of being obese, this comorbidity may impact how quickly the patient is able to recover from a knee replacement surgery but may not impact how quickly the patient is able to recover from a broken wrist. When steps 410 is executed for this patient, it may be determined how much the patient's obesity impacts his or her ability to recover from the knee replacement surgery and adjustment factor for the improvement score of the patient may be determined.

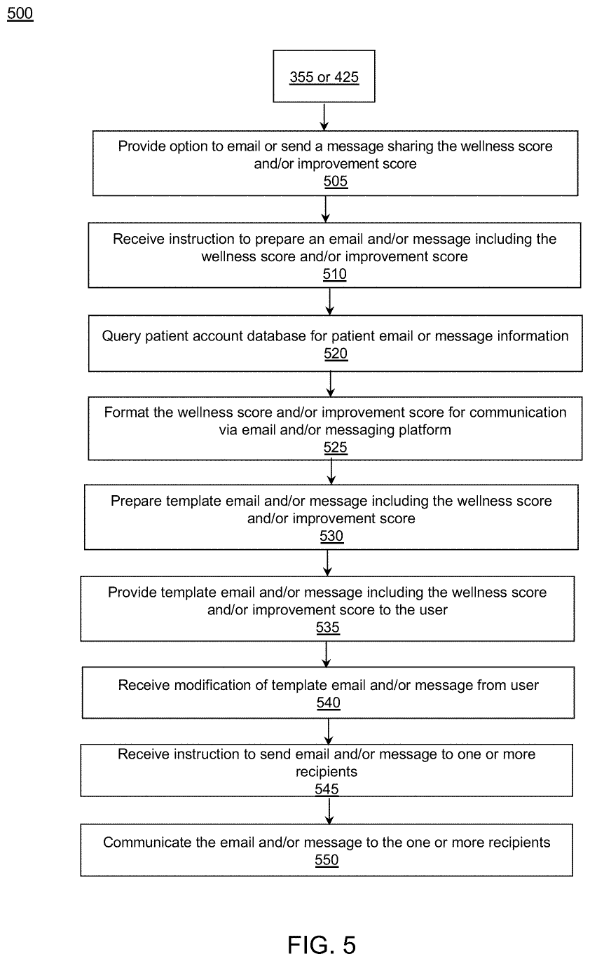

In step 420, an adjusted improvement score may be generated by applying the adjustment factor (of step 415) to the patient's improvement score. Then, in step 425, it may be determined whether the adjusted improvement score is above a threshold which may be the threshold of step 355 and/or a different threshold that incorporates the adjustment factor of step 415 and/or the comorbidity of the patient. When the adjusted improvements score is above the threshold, process 400 may advance to process 600, which is shown in FIGS. 6A and 6B and described below. Whether or not the adjusted improvement score is above the threshold, process 400 may also advance to process 500, which is shown in FIG. 5 and described below.

Whether, or not, the improvement score is below the threshold (or cannot be determined) (step 355), the user may be provided with an option to share his or her wellness score and/or improvement score via a message (e.g., SMS text message or message sent via a messaging app) and/or an email in step 505 as shown in FIG. 5, which provides a flowchart of an optional process 500 that may be executed following a determination in step 355 that the improvement score is below the threshold. An example of how the option to share a wellness and/or improvement score with another party is provided by graphic user interface 803 which has an icon 845, which when selected may allow the user to email or message another party with his or her wellness and/or improvement score and/or prompt the processor to prepare a template email according to one or more user and/or patient preferences.

In step 510, an instruction to prepare an email and/or message may be received via, for example receiving an indication that the user has selected icon 845. Optionally, in step 520, a memory storing data pertaining to the patient account and/or patient account database may then be queried (step 520) for any email and/or message preferences such as preferred addressees, email addresses, phone numbers, etc. associated with the patient. Once the requested email and/or message preferences are received, the wellness and/or improvement scores may be formatted for communication via email and/or the preferred messaging platform (step 525) and a template email and/or message including the wellness and/or improvement scores may be prepared (step 530) and communicated to the user (step 535). When step 520 is not performed, a template email and/or message including the wellness and/or improvement scores may be prepared (step 530) using a format-neutral text such as a rich text format document that may be copied and pasted into a message and/or email by the user. The format-neutral text and/or template email may then be communicated to the user (step 535).

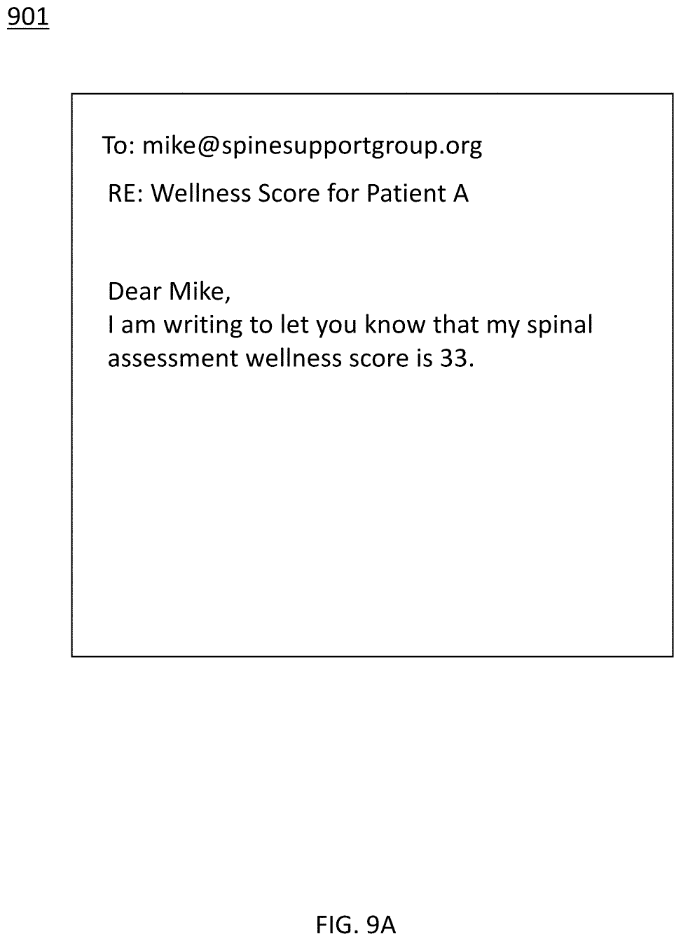

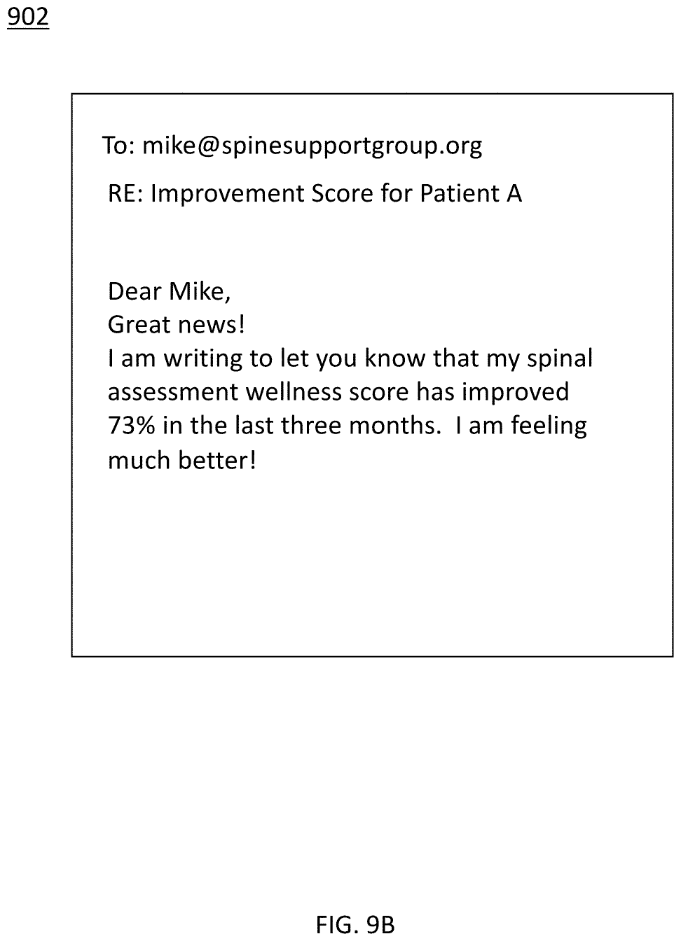

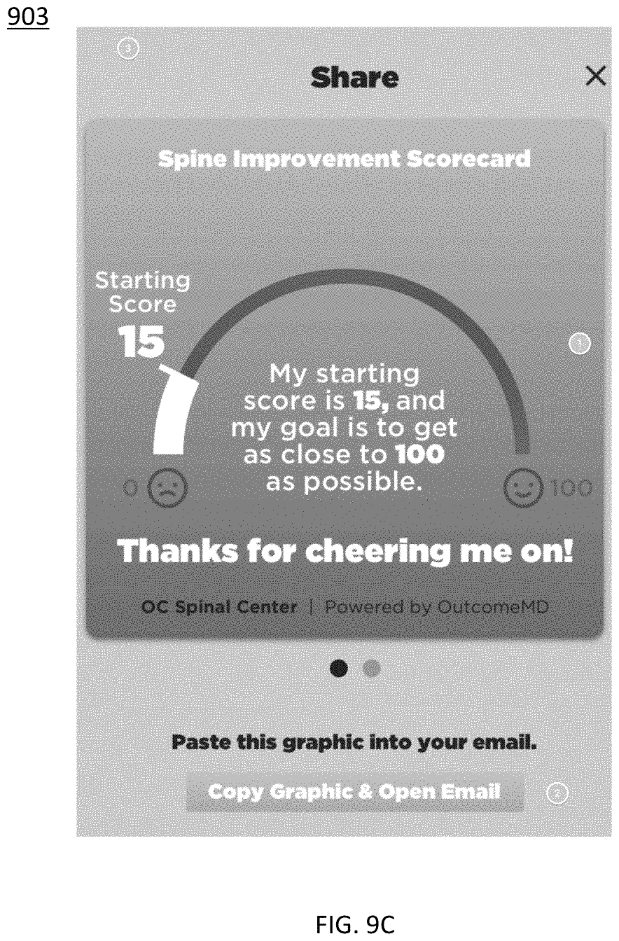

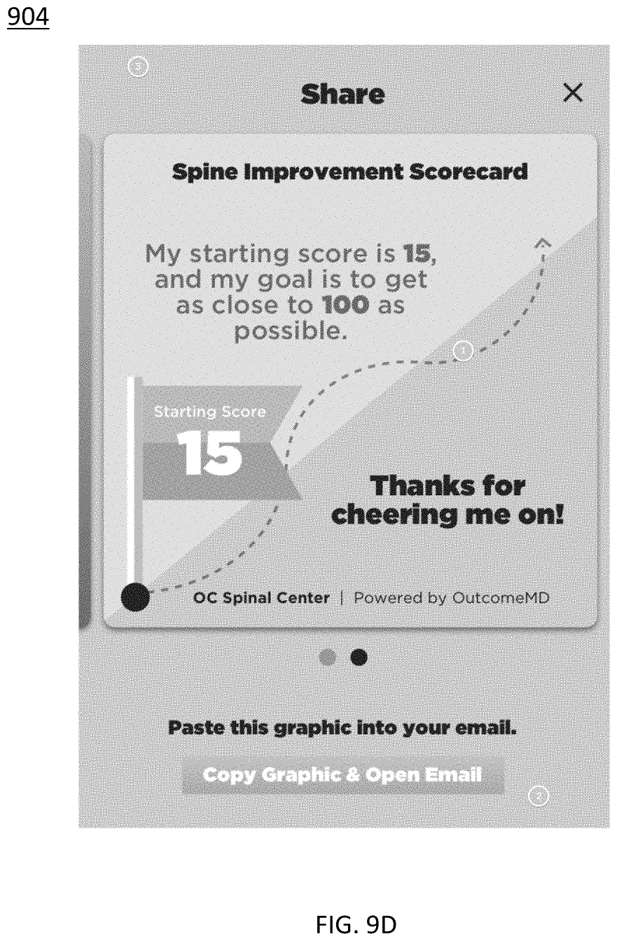

An exemplary template email that includes an improvement score is provided by FIG. 9B, which provides a user interface 902 showing a template email prepared for the user using, for example, one or more of the patient's email preferences which may, for example, be associated with the patient's account and/or received from the user during, for example, execution of step 305. In the example of FIG. 9B, the processor has populated the user's/patient's preferred recipient email address into the addressee line and the salutation into a template email with a template subject line (in this case, “improvement score for patient A”), and a template email body, which states the type of assessment taken, the improvement score, and the time period over which the improvement occurred.

Optionally, in some embodiments, process 500 may be performed in an order different from what is shown in FIG. 5. In these embodiments, a template email and/or message including the wellness and/or improvement score may be prepared and provided to the user (steps 530 and 535) may be performed following execution of step 355. On some occasions, this template email or message content may be provided to the user as a pop-up window. An exemplary GUI 903 providing a pop-up window that includes template email content is shown in FIG. 9C, which provides an initial wellness score (no improvement score) along with a button that may facilitate copying of the pop-up window content so that it may be pasted into an email or message. Another exemplary GUI 904 providing a pop-up window that includes template email content is shown in FIG. 9D, which provides an initial wellness score and an improvement score along with a button that may facilitate copying of the pop-up window content so that it may be pasted into an email or message.

In some cases, execution of step 535 may trigger display of a template email and/or message and/or text document that the user may review and/or edit prior to sending the email and/or message to one or more intended recipients. An exemplary template email is shown in FIG. 9A, which provides a user interface 901 showing a template email prepared for the user using, for example, one or more of the patient's email preferences which may, for example, be associated with the patient's account and/or received from the user during, for example, execution of step 305. In the example of FIG. 9A, the processor has populated the user's/patient's preferred recipient email address into the addressee line and the salutation into a template email with a template subject line (in this case, “wellness score for patient A”), and a template email body, which states the type of assessment taken and the wellness score for that assessment. In some cases, the user may then modify the template to, for example, personalize the text of the email and/or subject line and/or add or modify intended recipients of the email. Additionally, or alternatively, the user may send the email via his or her email program.

Optionally, in step 540, a modification of the template email and or message may be received from the user. Exemplary modifications include the addition, deletion, and/or adjustment of the text, or look (e.g., addition of graphic elements such as emojis, pictures, and logos) of the message and/or email. Optionally, in step 545, an instruction to send the email and/or message may be received and the email and/or message may be communicated (step 550) to the one or more of the intended recipients via, for example, a web browser, an SMS protocol, and/or email program.

In some embodiments, execution of step(s) 525 and/or 530 may include preparing a static image file that may include, for example, one or more of a patient's wellness scores like wellness scores 807, a patient's improvement score like improvement score graphic element 822, a graphic that indicates improvement such as semi-circular graph 812, a diagnosis, a treatment, a message like message 819, and/or a date. Examples of what a static image file may include are provided by the GUIs disclosed herein. Additionally, or alternatively, execution of step(s) 525 and/or 530 may include removal of personally identifying information (e.g., name, procedure or treatment, gender, etc.) from the information used to generate the template email and/or static image file. This may be accomplished by de-identifying the information used to generate the template email and/or static image file. In some cases, the static image file and de-identified information may be sent to a processor such as message, social media post, and/or email generator 140 so that the template email may be generated and communicated to the user.

When the improvement score is above the threshold in step 355, the user may be provided with an option to share his or her wellness score and/or improvement score via, for example, a social media post, an online review, an endorsement, a message, and/or an email via, for example, execution of process 600 as shown in FIGS. 6A and 6B and discussed below. If the user elects to share the patient's wellness and/or improvement score via a message and/or email, steps 510-550 of process 500 may be executed.

In step 605 of process 600, the user and/or patient may be provided with an option to share, or publish, a wellness score, an improvement score, an endorsement, and/or a review with, for example, another party, social media account, website, and/or online review platform. In some instances, step 605 may be executed via automatically providing a pop-up window or GUI interface by which a user may, for example, enter an endorsement of, for example, a medical treatment provider, a medical treatment facility, a treatment, and/or a course of action (e.g., diet or exercise program)

An example of how the option to share a wellness and/or improvement score with another party, or social media platform, is provided by graphic user interface 805 of FIG. 8E, which has an array of icons 875 wherein each icon represents a different way to share the wellness and/or improvement score(s). In the example of GUI 805, array of icons 875 includes an icon for a first social account SM A 860A, an icon for a second social account SM B 860B, an icon for an online review platform 856, and an icon for email 870. On some occasions, an icon of array of icons 875 may include a graphic that represents the respective social media platform, online review platform, and/or email/messaging service. Exemplary social media platforms include, but are not limited to, TWITTER™, FACEBOOK™, LINKEDIN™, and INSTAGRAM™. Exemplary online review platforms include, but are not limited to, YELP™, GOOGLE REVIEWS™ and an online doctor finder review platform that may be provided by, for example, an insurance company or medical facility.

In some embodiments, icons and/or the social media/online review platforms included in array 875 may be responsive to, for example, the patient's/user's preferences and/or the treatment provider's preferences. For example, if a patient has a social media account with the first social media platform but not the second social media platform, then array 875 may be modified by the patient to remove the SM B 860B icon and, in some instances may be further modified to include an icon that represents a third social media account/platform.

In addition to providing array 875, graphic user interface 805 (which also pertains to a spine assessment like GUI 803) includes heading 811 and a second wellness score window 810B that provides the previously determined wellness score of 33 along with a currently determined wellness score of 82, both of which are displayed on a fourth semi-circular graph 812D. Second semi-circular graph 812A also includes a second improvement score graphic element 822B that is positioned above fourth semi-circular graph 812D that graphically depicts a range on the graph from the initial wellness score of 33 to today's score of 82. Second wellness score window 810A also displays a fourth message 819D positioned underneath fourth semi-circular graph 812D that includes the patient's improvement score of 83% as a numerical value.

Graphic user interface 805 also provides a fourth scatter plot graph 820D that includes a scatter plot showing wellness, or outcome scores on the Y-axis as a function of time on the X-axis measured in months that includes first point 825A and a second point 825B, which represents the wellness score determined today of 82 aligned on the X-axis with when the second wellness score was determined between December of 2017 and January of 2018. Second scatter plot graph 820D also includes first visual indicator 840A, a second visual indicator 840B graphically depicting that the patient had an MRI lab test in November of 2017, a third visual indicator 840C graphically depicting that the patient was proscribed acetaminophen in November of 2017, a fourth visual indicator 840D graphically depicting that the patient had blood pressure reading taken in December of 2017, and a fifth graphically depicting that the patient had an office visit in January of 2018.

FIG. 8F provides a GUI 806 that shows a window with a first, or initial, wellness score 807, a sixth semi-circular graph 812F with a sixth improvement score graphic element 822F that is superimposed on sixth semi-circular graph 812F, which graphically depicts a range on the graph 812F from the initial wellness score of 15 to today's score of 76. GUI 806 further provides a sixth message 810F that states the patient's improvement score, and a “thank your doctor” icon 850, the selection of which may be received in step 610 of process 600.

FIG. 8G provides a GUI 807 that provides a message 855 with the patient's improvement score (in this case 61%) along with a template message 864 by which the patient may thank, or endorse, his or her doctor (in this case Dr. Brown) by selecting an “endorse Dr. Brown” icon 858. GUI 807 further includes array 875.

In step 610, an instruction to publish the wellness score, the improvement score, an endorsement, and/or a review to one or more of a social media platform, an online review platform, and/or a website may be received. In some embodiments, step 610 may be executed upon receipt of an indication that a user has selected, for example, one or more icons from array of icons 875 and/or a thank your doctor icon like thank your doctor icon 850. Optionally, in step 615, information regarding the selected social media platform, online review platform, and/or website may be accessed and/or queried for so that a draft, or template, social media post, online review, and/or endorsement that may include the patient's wellness and/or improvement score may be prepared (step 620). Preparation of a template social media post, online review, and/or endorsement may be performed using, for example, an application programming interface (API) program associated with the respective social media, endorsement, and/or online review platform. In some cases, step 615 may be executed by querying a website or other data store that may store APIs that may be used to generate the template.

On some occasions, step 610 may be executed by querying the patient account database for any social media, email, online review platform, and/or message preferences such as preferred social media platforms, user names, addresses, email addresses, phone numbers, etc. associated with the patient account. Additionally, or alternatively, step 610 may be executed by querying the patient account and/or an associated treatment provider computer/database for any social media, email, and/or message preferences such as preferred social media platforms, user names, addresses, email addresses, phone numbers, etc. associated with the treatment provider. The template social media post, online review, endorsement may then be provided to the user/patient (step 625) so that the user/patient may, for example, approve the template as-is or make modifications thereto.

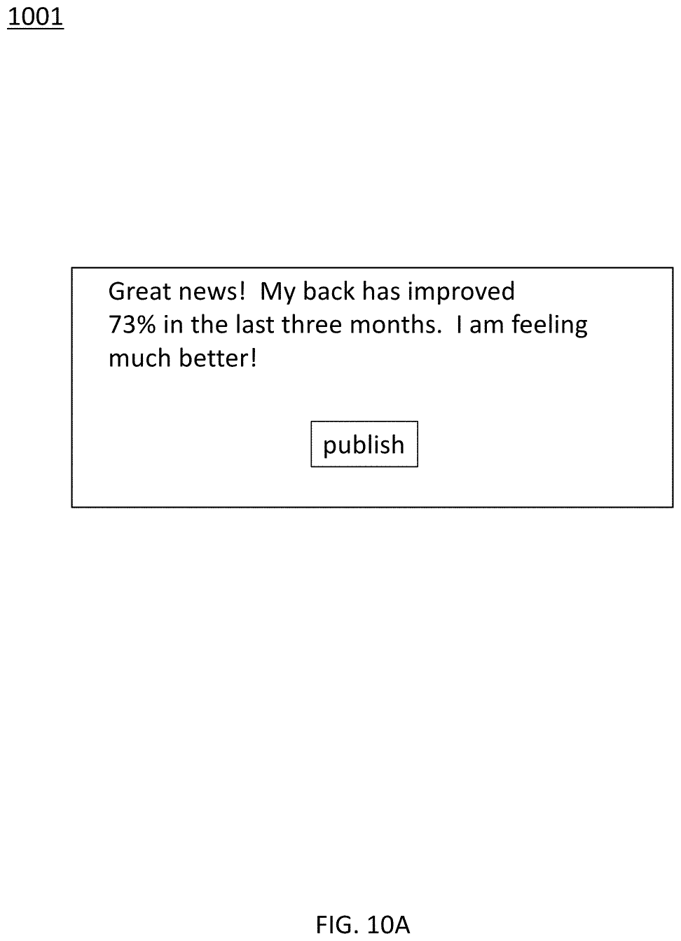

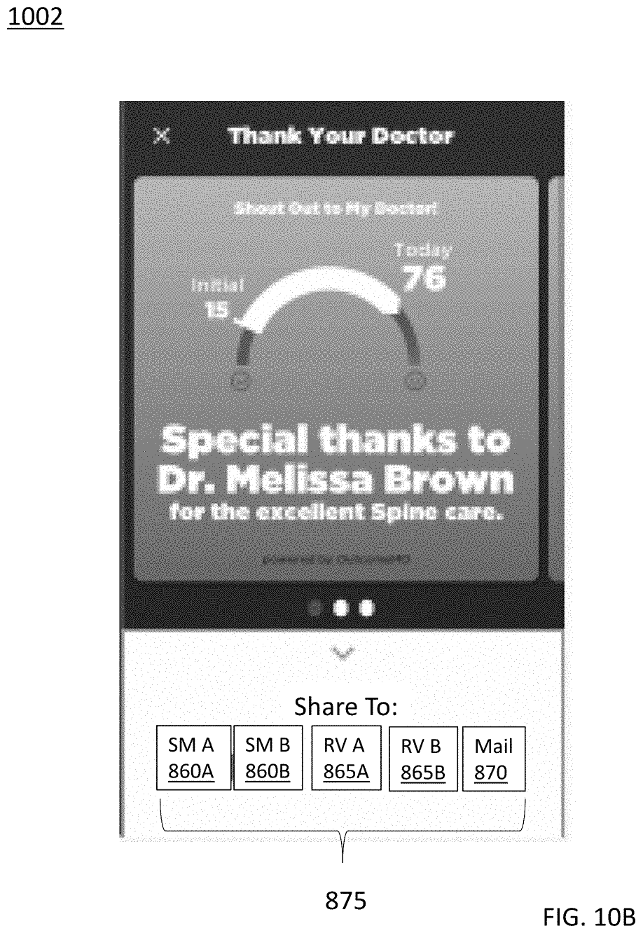

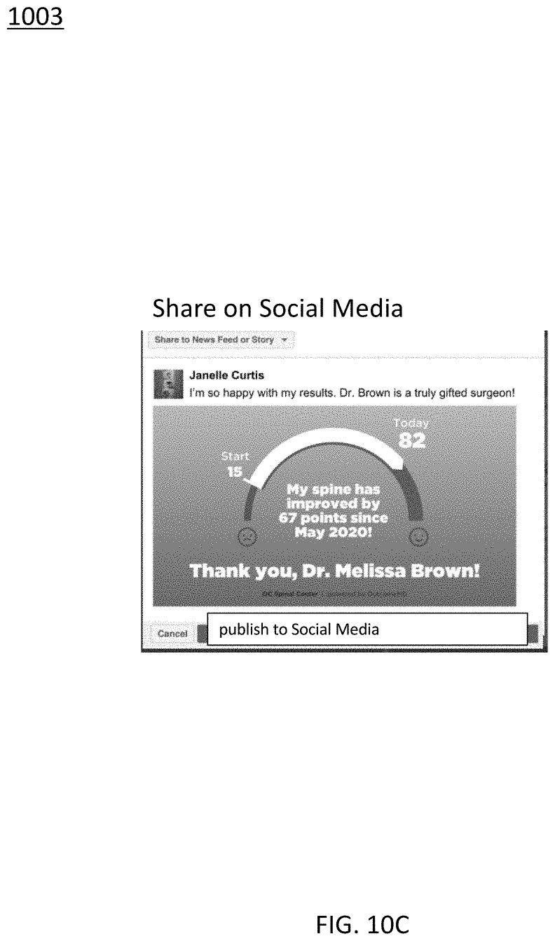

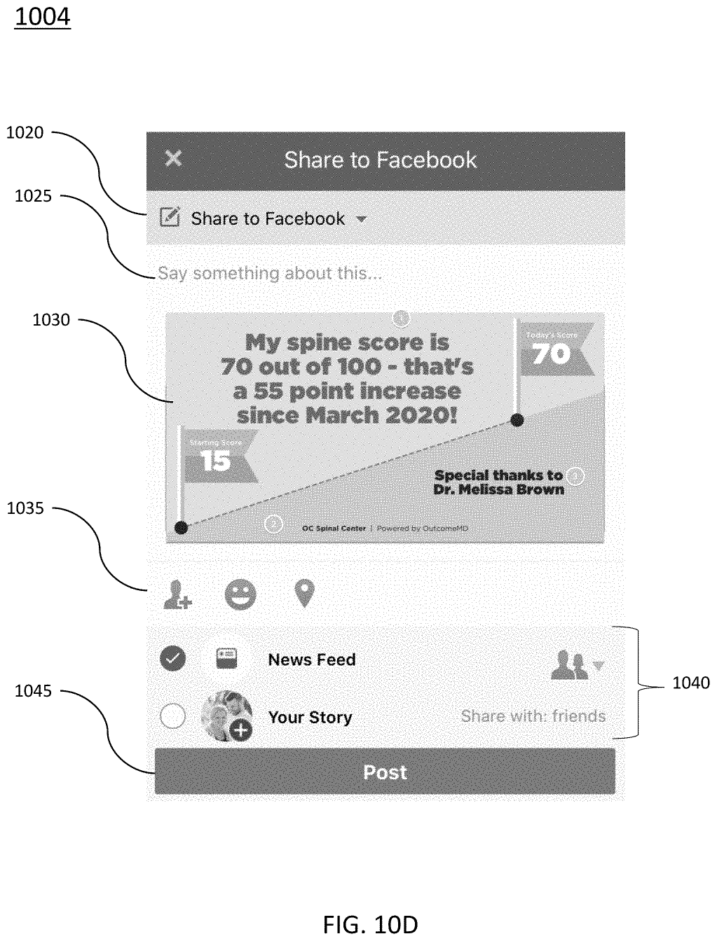

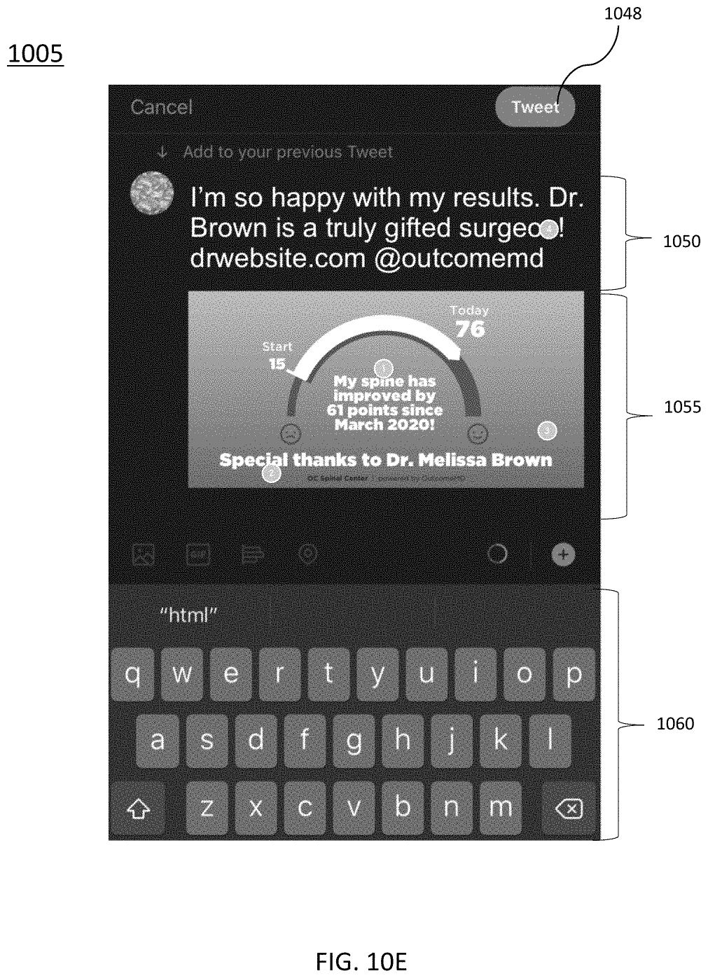

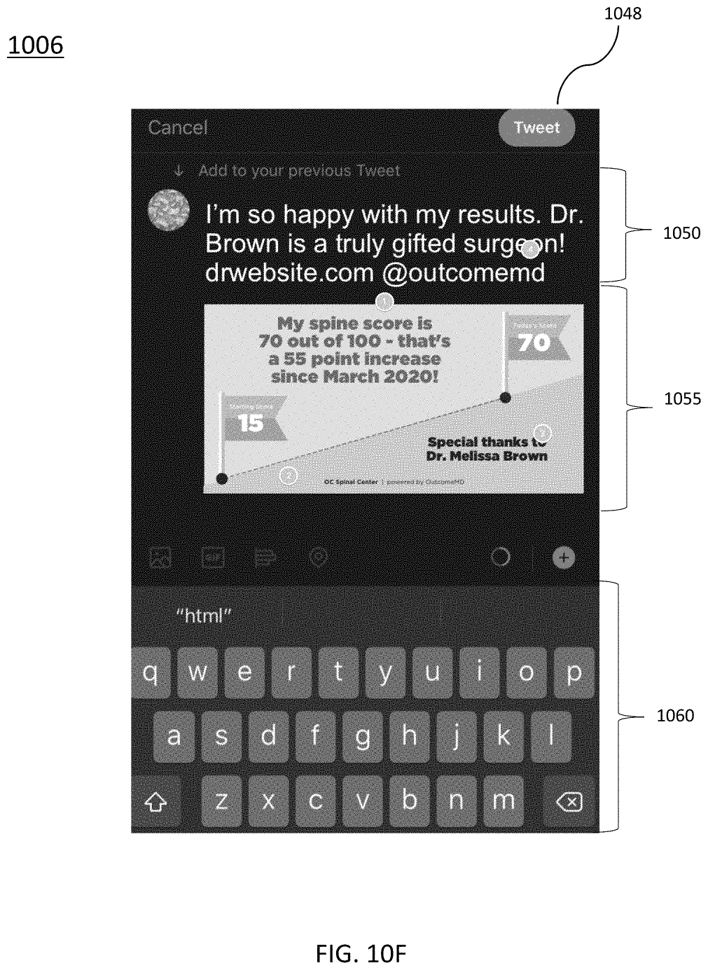

An exemplary template social media post that may be provided to the user in step 625 is shown in FIG. 10A, which provides an exemplary GUI 1001 that includes a draft social media post 1001 that includes some template text (in this case “Great news! My back has improved 73% in the last three months. I am feeling much better!”). The user may then edit draft social media post 1000 and post it via, for example, selection of the publish icon provided by GUI 1001. FIG. 10B provides a GUI 1002 that provides another exemplary template social media post that includes both wellness and improvement scores that a user may share via, for example, selection of one or more icons provided by array 875. FIG. 10C provides a GUI 1003 that provides another exemplary template social media post that includes both wellness and improvement scores along with a message thanking the patient's doctor (in this case, Dr. Melissa Brown) that a user may share via, for example, selection of one or more icons provided by array 875. The patient/user may publish the template to, for example, a social media platform and/or online review platform via selection of the publish icon of GUI 1001, selection of one or more icons provided by array 875 provided by GUI 1002, and/or a publish to social media icon provided by GUI 1003.

FIG. 10D provides a social media (in this case, Facebook) post template GUI 1004 where the user may provide instructions for posting his or her wellness score. GUI 1004 includes a window 1030 that displays two wellness scores of the patient determined at different times along with an endorsement of the user's doctor in the form of a message that states “special thanks to Dr. Melissa Brown”. GUI 1004 also provides a text box 1025 in which the user may enter text or other information (e.g., emojis or symbols) via selection of a keyboard interface activation menu 1035, a dropdown menu 1020 that provides the user with one or more social media platforms to which to post information, a menu of places to publish the social media post that may be selected by, for example, selecting a radio button adjacent to a social media account and/or online review platform to post the message including the wellness and/or improvement scores. GUI 1004 may further contain a button 1045 (in this case labeled “Post”) by which to communicate the message/social media post to the social media platform.

FIG. 10E provides a social media template GUI 1005 formatted for posting on a social media platform like TWITTER™ that includes a window showing two wellness scores and a range of improvement between the wellness scores. GUI 1005 also includes a keyboard by which a user may enter a message, in this case, “I′m so happy with my results. Dr. Brown is a truly gifted surgeon!” which is displayed in message display window 1050. Also present in message display window 1050 is a website for the doctor for the user (in this case, Dr. Brown) in the form of text that says “drwebsite.com”. GUI 1006 of FIG. 10F is similar to GUI 1005 with the exception that the content of window 1055 is displayed differently so that instead of displaying the wellness scores on a semi-circular graph (as in GUI 1005), the wellness scores are displayed on scatter point graph.

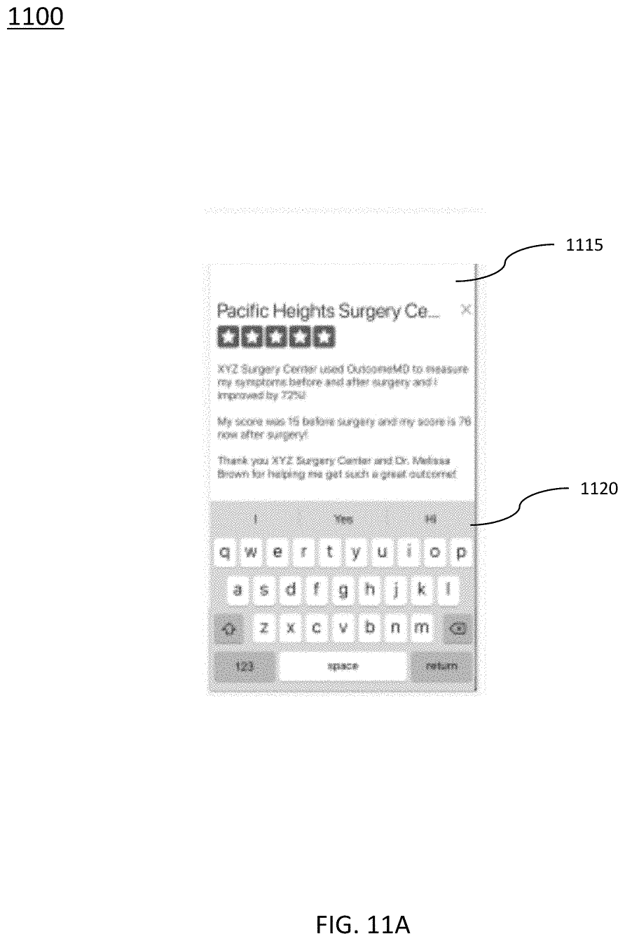

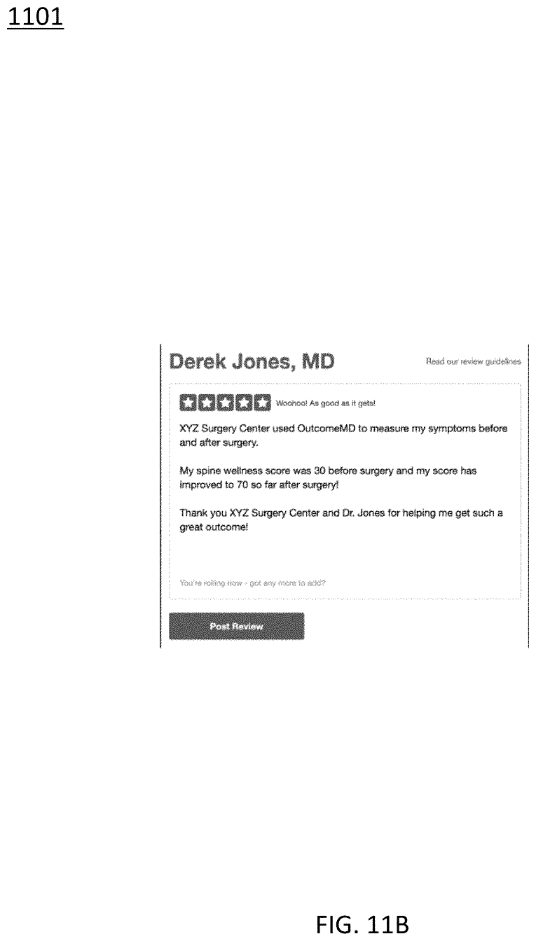

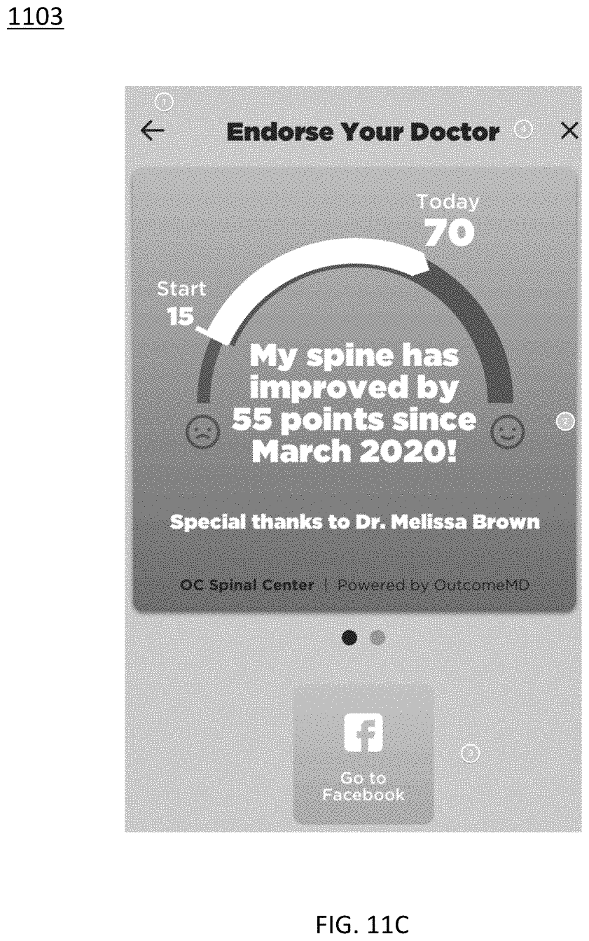

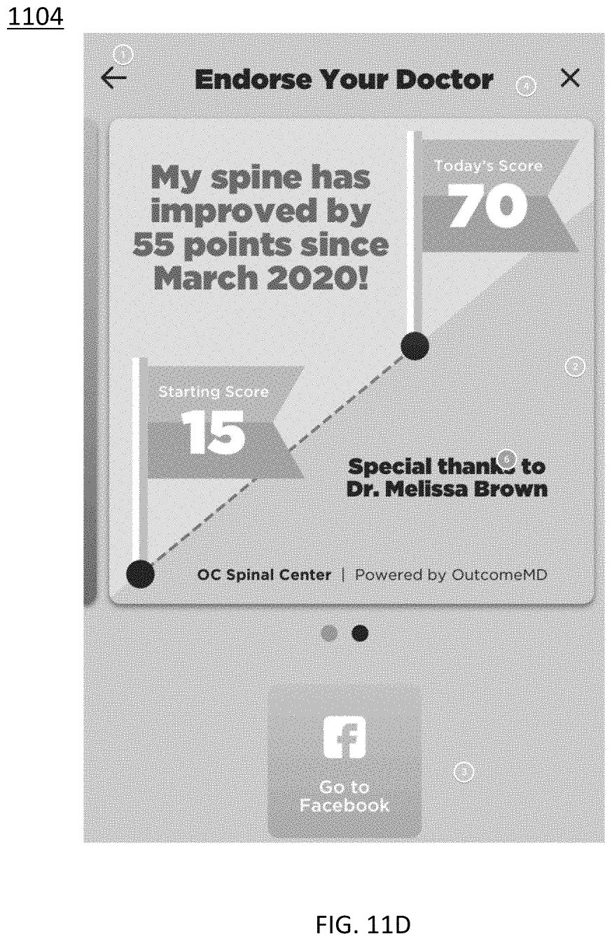

FIGS. 11A and 11B provide exemplary GUIs 1100 and 1101, respectively, that provide template online reviews that may be provided to the user/patient in step 625. GUI 1100 provides a window 1115 that shows a 5-star rating and text of a review to be posted to an online review platform. GUI 1100 further provides an interface 1120 by which the user may modify the template message (via typing on a touch-sensitive portion of the GUI). FIG. 11B provides a GUI 1101 provides a window that shows a complete online review that includes a 5-star rating and text of a review to be posted to an online review platform along with a post review icon. GUI 1101 does not display interface 1120 because the patient has completed preparing the review. FIGS. 11C and 11D provide endorsement GUIs 1103 and 1104, respectively, that provide a template endorsement for Dr. Melissa Brown that may be posted to an online review platform and/or a social media platform according to, for example, one or more processes described herein.

Optionally, in step 630, a modification of the template email, social media post, review, endorsement, and/or message may be received from the user. Exemplary modifications include the addition, deletion, and/or adjustment of the text, or look (e.g., text color or formation and/or an addition of graphic elements such as emojis, pictures, and logos) of the email, social media post, review, endorsement, and/or message. In step 635, an instruction to communicate the email, social media post, review, endorsement, and/or message to a social media platform, an email platform or server, an online review platform, and/or a messaging platform may be received and an acknowledgement that the email, social media post, review, endorsement, and/or message has been communicated, posted, and/or published to the respective social media platform, social media account, email platform or server, online review platform, online review platform account, messaging account, and/or messaging platform may, in some cases, be provided to the user. The email, social media post, review, endorsement, and/or message may then be communicated to the relevant social media platform, social media account, email platform or server, online review platform, online review platform account, messaging account, and/or messaging platform (step 640). In some embodiments, preparing the email, social media post, review, endorsement, and/or message for publication and/or communication may include associating enriched data (e.g., metadata, hyperlinks, and/or sets of instructions for reporting, for example, views of and/or clicks on the published improvement score and/or or review) with the email, social media post, review, endorsement, and/or message so that, for example, future activity related to the email, social media post, review, endorsement, and/or message may be tracked, reported, and/or measured.

Optionally, an acknowledgement that the email, social media post, review, endorsement, and/or message was successfully communicated and/or published by the relevant social media platform, social media account, email platform or server, online review platform, online review platform account, messaging account, and/or messaging platform may be received (step 645) and that acknowledgement and/or a confirmation message may be communicated to the user (step 650)

In some embodiments, a sender of the medical questionnaire may be invoiced or charged for some, or all, of the emails, social media posts, reviews, endorsements, and/or messages the user/patient publishes as part of, for example, an online reputation management and/or marketing campaign. In step 655, an indication that the email, social media post, review, endorsement, and/or message has been published to, for example, a social media platform, social media account, email platform or server, online review platform, online review platform account, messaging account, and/or messaging platform may be received. It may then be determined whether publication of the email, social media post, online review, endorsement, and/or message is subject to a fee or is associated with a reputation management and/or marketing campaign of, for example, the sender of a medical questionnaire from which the improvement score may be derived, a treatment provider, and/or treatment facility (step 660). When the published email, social media post, review, endorsement, and/or message is not subject to a fee or associated with a reputation management and/or marketing campaign of the sender, process 600 may end.

When the published email, social media post, review, endorsement, and/or message is subject to a fee or is associated with a reputation management and/or marketing campaign of, for example, the sender of a medical questionnaire from which a wellness score and/or improvement score may be derived, a treatment provider, and/or treatment facility, one or more characteristics of the published email, social media post, review, endorsement, and/or message may be determined (step 665). Exemplary characteristics include, but are not limited to, characterisitics (e.g., number of followers and/or viewers exposed to the email, social media post, review, endorsement, and/or message; a geographic and/or demographic reach of the social media platform, social media account, email platform or server, online review platform, online review platform account, messaging account, and/or messaging platform; and/or demographic information of followers and/or viewers exposed to the email, social media post, review, endorsement, and/or message) of the social media platform, social media account, email platform or server, online review platform, online review platform account, messaging account, and/or messaging platform the improvement score, email, social media post, review, endorsement, and/or message is published to, the online review platform the improvement score and/or online review is published to, a degree of complexity to preparing and/or communicating the email, social media post, review, endorsement, and/or message, a characteristic of the medical questionnaire, a characteristic of the user, a characteristic of the sender, a time of year email, social media post, review, endorsement, and/or message is published, a characteristic of the patient/user, a characteristic of the treatment administered to the patient, a characteristic of a diagnosis of the patient, a comorbidity of the patient, and/or a characteristic (e.g., complexity) of how a wellness score and/or improvement score is calculated. In some embodiments, step 665 may be performed by analzying the published improvement score and/or or review to determine one or more properties thereof such as whether the published improvement score and/or or review is associated with enriched data (e.g., metadata, hyperlinks, and/or sets of instructions for reporting, for example, views of and/or clicks on the published improvement score and/or or review).

Then, in step 670, a fee for the published email, social media post, review, endorsement, and/or message may be determined, the sender may then be charged the fee (step 675), and provided with an indication (e.g., invoice and/or receipt) that their account has been charged (step 680).

In some embodiments, execution of step(s) 525, 530, 625 and/or 630 may include preparing a static image file that may include, for example, one or more of a patient's wellness scores like wellness scores 807, a patient's improvement score like improvement score graphic element 822, a graphic that indicates improvement in wellness scores such as semi-circular graph 812, a diagnosis, a treatment, a message like message 819, and/or a date. Examples of what a static image file may include are provided by the GUIs disclosed herein. Additionally, or alternatively, execution of step(s) 525, 530, 625 and/or 630 may include removal of personally identifying information (e.g., name, procedure or treatment, gender, etc.) from the information used to generate the template email and/or static image file. This may be accomplished by de-identifying the information used to generate the template email and/or static image file. In some cases, the static image file and de-identified information may be sent to a processor such as message, social media post, and/or email generator 140 so that the template email may be generated and communicated to the user.

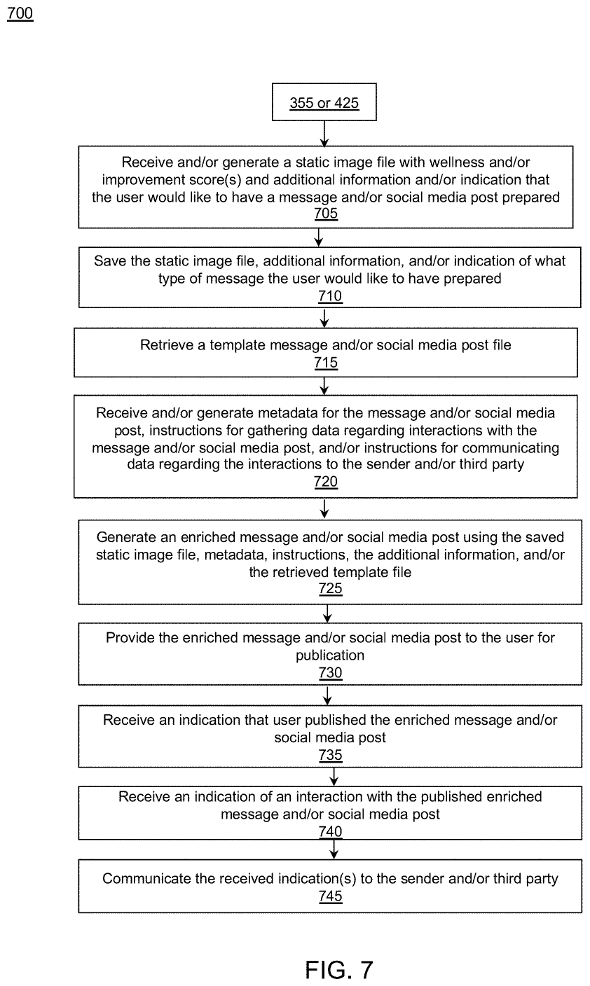

FIG. 7 provides a flowchart illustrating an exemplary process 700 for preparing, for example, an enriched social media post, online review, and/or message. Process 700 may be executed by, for example, any of the systems and/or system components disclosed herein. In some embodiments, process 700 may be executed following execution of step 355425, 530, and/or 620.

In step 705, a static image file that includes the wellness score and/or improvement score may be received and/or generated. In some embodiments, additional information (e.g., treatment the patient is undergoing, patient diagnosis, how long since a treatment was administered, patient/user characteristics, a treatment provider, and/or a medical facility, etc.) may also be received in step 705. Additionally, or alternatively, an indication that the user would like to have a message and/or social media post prepared may be received in step 705. This indication may include, for example, a type of social media post, a type of message, and/or a preferred social media/messaging platform the patient, a sender of a medical questionnaire from which a wellness and/or improvement score is derived, and/or other user prefers and/or requires.

In step 710, the static image file, additional information, and/or indication of patient, sender, and/or user preferences and/or requirements what type of message the user would like to have prepare may be saved. In step 715, a template email, social media post, review, endorsement, and/or message and/or information that may be used to generate a email, social media post, review, endorsement, and/or message may be retrieved from, for example, a database like template database 141 responsively to, for example, the indication of what type of email, social media post, review, endorsement, and/or message the patient, sender, and/or user would like generated, a characteristic of the medical questionnaire the patient has answered, a characteristic of the patient, a characteristic of the sender of the medical questionnaire from which the wellness and/or improvement score is derived, a treatment associated with the patient, and/or a characteristic of a treatment facility for the patient. The template email, social media post, review, endorsement, and/or message file and/or information used to generate a email, social media post, review, endorsement, and/or message may include, for example, a format for the email, social media post, review, endorsement, and/or message, content (e.g., treatment provider name, treatment received by the patient, a logo, etc.) to be included in the email, social media post, review, endorsement, and/or message, instructions for how to incorporate information included in and/or derived from the medical questionnaire and/or answers thereto into the email, social media post, review, endorsement, and/or message, metadata, instructions for gathering data regarding an interaction with a published email, social media post, review, endorsement, and/or message, and/or instructions for communicating data regarding the interactions with the email, social media post, review, endorsement, and/or message to the sender and/or third party.

In step 720, metadata for the email, social media post, review, endorsement, and/or message may be received and/or generated. Additionally, or alternatively, execution of step 720 may include receiving and/or generating instructions for gathering data regarding interactions with the email, social media post, review, endorsement, and/or message once it is published, and/or instructions for communicating data regarding the interactions to the sender of the medical questionnaire and/or a third party (e.g., a third party operator of server 102). The metadata, instructions for gathering data, and/or instructions for communicating data via execution of step 720 may be responsive to, for example, the template/information of step 715 one or more preferences of, for example, the sender, the third party, or another user that may be received in step 705. In some embodiments, the enriched data, metadata, and/or instructions associated with an enriched email, social media post, review, endorsement, and/or message and/or static image file included thereinmay be in the form of HTML or other executable computer software code.

In some embodiments, the metadata generated and/or retrieved for inclusion in an enriched message and/or social media post may include, for example, keywords relating to a condition of the patient, a treatment received by the patient, a diagnosis of the patient, a specialty or focus area of the sender of the medical questionnaire and/or treatment provider, a sub-specialty of the sender and/or treatment provider, demographic information for the sender and/or treatment provider, demographic and/or comorbidity information for the patient, business information (e.g., location, practice size, board accreditations, certifications, languages spoken, etc.) for the sender and/or treatment provider, contact information (e.g., phone number, website address, and/or email address) for the sender/treatment provider, and/or social media accounts associated with the sender/treatment provider. At times, the metadata may be optimized for access by internal systems (e.g., indexes of data within an organization) and/or external systems such as search engines like GOOGLE™ or BING™ so that, for example, a placement of the social media post, online review, endorsement, and/or message is optimized within a set of search results returned in response to a search query including the keyword.

In some embodiments, the metadata and/or instructions of step 720 may include links to, for example, a website, a social media account, and/or an online review account of, for example, a sender of the medical questionnaire, a treatment provider, a treatment facility, and/or a third party. Additionally, or alternatively, the metadata and/or instructions of step 720 may include links to, for example, to social media and/or online review or ratings platforms such as YELP™ and/or GOOGLE REVIEWS™. In some embodiments, the metadata and/or instructions of the enriched message and/or social media post may be optimized for sharing on one more social media platforms using metadata that social media platforms utilize to maximize sharing within and/or searching optimization on their platforms.

In step 725, an enriched email, social media post, review, endorsement, and/or message may be generated using the saved static image file of step 710, the template retrieved in step 715, and/or the metadata and/or instructions of step 720. The enriched email, social media post, review, endorsement, and/or message may then be provided to, for example, the user or patient (step 730) for publication by the user or patient to, for example, his or her social media platform, social media account, email platform or server, online review platform, online review platform account, messaging account, and/or messaging platform.

Optionally, in step 735, an indication (e.g., acknowledgement) that the email, social media post, review, endorsement, and/or message has published by and/or been communicated to social media platform, social media account, email platform or server, online review platform, online review platform account, messaging account, and/or messaging platform may be received. In some cases, when the email, social media post, review, endorsement, and/or message is published to a social media platform, social media account, email platform or server, online review platform, online review platform account, messaging account, and/or messaging platform operated and/or managed by the sender and/or a third party, this indication may be the appearance of the published message and/or social media post on the social media platform, social media account, email platform or server, online review platform, online review platform account, messaging account, and/or messaging platform operated by the sender and/or a third party. In other embodiments, the indication may be a communication executed per the instructions of step 725 that are associated with the enriched email, social media post, review, endorsement, and/or message. For example, if the instructions of step 725 include instructions to send a message and/or acknowledgement notification to the sender of the medical questionnaire and/or third party when the enriched email, social media post, review, endorsement, and/or message is published then this message/acknowledgement notification may be received in step 735 for eventual communication to the sender/third party via execution of step 745.

Optionally, in step 740, an indication of an interaction with the published message and/or social media post may be received. Exemplary interactions include, but are not limited to, views of the message and/or social media post and actions (e.g., clicks, selection of a link, entry of text into a text box, and/or selection of an icon) taken within/on the message and/or social media post by the user and/or other viewers of the message and/or social media post. The indication may be a communication executed per the instructions of step 725 that are associated with the enriched email, social media post, review, endorsement, and/or message. For example, if the instructions of step 725 include instructions to send a message and/or acknowledgement notification to the sender of the medical questionnaire and/or third party when the enriched email, social media post, review, endorsement, and/or message is interacted (e.g., viewed and/or clicked on) with, then this indication may be communicated to the sender/third party via execution of step 735 when the interaction occurs.

In step 745, the indication(s) of steps 735 and/or 740 may be communicated to the sender and/or third party via, for example, email, posting on a website, or in another preferred format (e.g., a report) for the sender and/or third party. On some occasions, the sender and/or third party may be billed for receipt of the indications of steps 735 and/or 740 on, for example, a per view and/or per click basis in, for example, a manner similar to that described above with regard to process 600.

In some embodiments, execution of process(es) 300, 400, 500, 600, and/or 700 may serve to offer the medical questionnaire sender and/or third party greater control of their respective reputation on social media, online review, and/or message platforms by directing a user to create a social media post and/or message that accurately captures, for example, the patient's condition and/or improvement following treatment. Additionally, or alternatively, execution of process(es) 300, 400, 500, 600, and/or 700 may serve to offer the medical questionnaire sender and/or third party greater visibility into what their patients are saying online about them. Additionally, or alternatively, execution of process(es) 300, 400, 500, 600, and/or 700 may serve to enable the medical questionnaire sender and/or third party to track visitors to a social media post and/or message platform and/or account, which may, in some cases, inform a marketing and/or online reputation strategy.

FIG. 12 is a block diagram showing a system 1200 includes a bus 1202 or other communication mechanism for communicating information, and a processor 1204 coupled with the bus 1202 for processing information. Computer system 1200 also includes a main memory 1206, such as a random-access memory (RAM) or other dynamic storage device, coupled to the bus 1202 for storing information and instructions to be executed by processor 1204. Main memory 1206 also may be used for storing temporary variables or other intermediate information during execution of instructions to be executed by processor 1204. Computer system 1200 further includes a read only memory (ROM) 1208 or other static storage device coupled to the bus 1202 for storing static information and instructions for the processor 1204. A storage device 1210, for example a hard disk, flash memory-based storage medium, or other storage medium from which processor 1204 can read, is provided and coupled to the bus 1202 for storing information and instructions (e.g., operating systems, applications programs and the like).

Computer system 1200 may be coupled via the bus 1202 to a display 1212, such as a flat panel display, for displaying information to a computer user. An input device 1214, such as a keyboard including alphanumeric and other keys, may be coupled to the bus 1202 for communicating information and command selections to the processor 1204. Another type of user input device is cursor control device 1216, such as a mouse, a track pad, or similar input device for communicating direction information and command selections to processor 1204 and for controlling cursor movement on the display 1212. Other user interface devices, such as microphones, speakers, etc. are not shown in detail but may be involved with the receipt of user input and/or presentation of output.

The processes referred to herein may be implemented by processor 1204 executing appropriate sequences of computer-readable instructions contained in main memory 1206. Such instructions may be read into main memory 1206 from another computer-readable medium, such as storage device 1210, and execution of the sequences of instructions contained in the main memory 1206 causes the processor 1204 to perform the associated actions. In alternative embodiments, hard-wired circuitry or firmware-controlled processing units may be used in place of or in combination with processor 1204 and its associated computer software instructions to implement the invention. The computer-readable instructions may be rendered in any computer language.

In general, all of the above process descriptions are meant to encompass any series of logical steps performed in a sequence to accomplish a given purpose, which is the hallmark of any computer-executable application. Unless specifically stated otherwise, it should be appreciated that throughout the description of the present invention, use of terms such as “processing”, “computing”, “calculating”, “determining”, “displaying”, “receiving”, “transmitting” or the like, refer to the action and processes of an appropriately programmed computer system, such as computer system 1200 or similar electronic computing device, that manipulates and transforms data represented as physical (electronic) quantities within its registers and memories into other data similarly represented as physical quantities within its memories or registers or other such information storage, transmission or display devices.

Computer system 1200 also includes a communication interface 1218 coupled to the bus 1202. Communication interface 1218 may provide a two-way data communication channel with a computer network, which provides connectivity to and among the various computer systems discussed above. For example, communication interface 1218 may be a local area network (LAN) card to provide a data communication connection to a compatible LAN, which itself is communicatively coupled to the Internet through one or more Internet service provider networks. The precise details of such communication paths are not critical to the present invention. What is important is that computer system 1200 can send and receive messages and data through the communication interface 1218 and in that way communicate with hosts accessible via the Internet. It is noted that the components of system 1200 may be located in a single device or located in a plurality of physically and/or geographically distributed devices.

We claim:

1. A computer-implemented method comprising:
providing, by the processor, a medical questionnaire to a patient via a first graphic user interface displayed on a display device, the medical questionnaire being associated with a sender and a medical condition and a scoring procedure for scoring responses to the medical questionnaire;
receiving, by the processor, a set of responses to the medical questionnaire from the patient via the first graphic user interface;
determining, by the processor, a wellness score for the patient by applying the scoring procedure to the set of responses;
receiving, by the processor, a previously determined wellness score for the patient;
comparing, by the processor, the determined wellness score and a received previously determined wellness score for the patient;
determining, by the processor, an improvement score using a result of the comparing;
determining, by the processor, whether the improvement score is above a threshold for improvement scores;
providing, by the processor, the patient with a second graphic user interface by which the patient may provide an instruction to post the improvement score to an account on a social media platform responsively to a determination that the improvement score is above a threshold value for improvement scores;
receiving, by the processor, the instruction to post the improvement score on the social media platform via the second graphic user interface;
formatting, by the processor, the improvement score for posting to the account on the social media platform thereby generating a formatted social media post that includes the improvement score; and
communicating, by the processor, the formatted social media post that includes the improvement score to the social media platform.

2. The method of claim 1, wherein the account on the social media platform is a social media account of the patient.

3. The method of claim 1, wherein the account on the social media platform is a private social media account of the patient.

4. The method of claim 1, wherein the account on the social media platform is a social media account of the sender.

5. The method of claim 1, wherein the medical questionnaire is embedded with metadata that facilitates communication with the social media platform.

6. The method of claim 1, wherein the second graphic user interface is embedded with metadata that facilitates communication with the social media platform.

7. The method of claim 1, wherein the second graphic user interface includes a plurality of social media posting options, the method further comprising:
receiving, by the processor, an indication of a social media preference of the patient, wherein the plurality of social media posting options provided by the second graphic user interface are responsive to the received social media preference of the patient.

8. The method of claim 1, wherein the second graphic user interface includes a plurality of social media posting options, the method further comprising:
receiving, by the processor, an indication of a social media preference of a treatment provider who administered the medical treatment to the patient, wherein the plurality of social media posting options provided by the second graphic user interface are responsive to the received social media preference of the treatment provider.

9. The method of claim 8, wherein the social media preference of the treatment provider is associated with the medical questionnaire.

10. The method of claim 1, wherein the previously determined wellness score was determined by applying the scoring procedure to a set of previously received responses to the medical questionnaire, the set of previously received responses to the medical questionnaire being received prior to the patient receiving the treatment.

11. The method of claim 1, further comprising:
receiving, by the processor, a characteristic of the patient; and
determining, by the processor, whether the patient characteristic impacts the determination of at least one of the wellness score and the improvement score and, if so, adjusting the at least one wellness score and the improvement score using the patient characteristic, wherein the determination of whether the improvement score is above the threshold for improvement scores includes determining whether the adjusted improvement score is above a threshold for adjusted improvement scores.

12. The method of claim 11, wherein the patient characteristic is a comorbidity.

13. The method of claim 1, wherein the threshold is specific to at least one of the treatment and a provider of the treatment.

14. The method of claim 1, further comprising:
providing, by the processor, the patient with a third graphic user interface by which the patient may provide an instruction to post the improvement score to an online review platform responsively to a determination that the improvement score is above a threshold for improvement scores;
receiving, by the processor, the instruction to post the improvement score to the account on the online review platform;
formatting, by the processor, the improvement score for posting to the online review platform; and
communicating, by the processor, the improvement score to the online review platform.

15. The method of claim 1, further comprising:
providing, by the processor, a template social media post including the improvement score to the patient prior to posting the improvement score on the social media platform; and
receiving, by the processor, an instruction to post the template social media post to the social media platform, wherein the improvement score is posted on the social media platform using the template social media post.

16. The method of claim 1, further comprising:
providing, by the processor, a template social media post including the improvement score to the patient prior to posting the improvement score on the social media platform;
receiving, by the processor, a modification to the template social media post;
generating, by the processor, a modified social media post responsively to the received modification; and
receiving, by the processor, an instruction to communicte the modified social media post to the social media platform.

17. The method of claim 1, further comprising:
receiving, by the processor, an indication that the patient has scheduled the treatment prior to provision of the medical questionnaire to the patient via the first graphic user interface,
wherein provision of the medical questionnaire to the patient is responsive to receiving the indication that the patient has scheduled the treatment.

18. The method of claim 1, further comprising:
receiving, by the processor, an indication that the patient has scheduled an encounter with the treatment provider who provided the treatment to the patient following provision of the treatment to the patient, wherein provision of the medical questionnaire to the patient is responsive to receiving the indication that the patient has scheduled the encounter.

19. The method of claim 1, wherein the formatting includes encoding the social media post with a hyperlink linking the social media post to a uniform resource locator (URL) associated with the sender of the medical questionnaire.

20. The method of claim 1, wherein the formatting includes encoding with metadata associating the social media post with the sender of the medical questionnaire.
